(19) United States
(12) Patent Application Publication
(10) Pub. No.: US 2024/0035060 A1
(43) Pub. Date: Feb. 1, 2024

(54) METHODS AND SYSTEMS FOR CELL-FREE BIODISCOVERY OF NATURAL PRODUCTS

(71) Applicant: SYNVITROBIO, INC., San Leandro, CA (US)

(72) Inventors: Zachary Z. Sun, San Leandro, CA (US); Richard Mansfield, San Leandro, CA (US); Abel C. Chiao, San Leandro, CA (US); Kelly S. Trego, San Leandro, CA (US)

(21) Appl. No.: 18/311,555

(22) Filed: May 3, 2023

Related U.S. Application Data
(63) Continuation of application No. 16/499,233, filed on Sep. 27, 2019, now abandoned.
(60) Provisional application No. 62/620,310, filed on Jan. 22, 2018.
(60) Provisional application No. 62/482,856, filed on Apr. 7, 2017.
(60) Provisional application No. 62/467,548, filed on Mar. 6, 2017.

(51) Int. Cl. C12P 21/02; C12P 19/34

(52) U.S. Cl. CPC C12P 21/02; C12P 19/34

(57) ABSTRACT

Provided herein, in one aspect, is a composition for in vitro transcription and translation, comprising: a treated cell lysate derived from one or more organisms such as bacteria, archaea, plant or animal; a plurality of supplements for gene expression; an energy recycling system for providing adenosine triphosphate and recycling adenosine diphosphate; and an engineered propeptide operably linked to a stabilizing domain. Methods for making and using the same are also provided.

### CROSS-REFERENCE TO RELATED APPLICATIONS

This application claims priority to and the benefit of U.S. Provisional Application Nos. 62/467,548 filed Mar. 6, 2017, 62/482,856 filed Apr. 7, 2017 and 62/620,310 filed Jan. 22, 2018, the entire disclosure of all of which is hereby incorporated by reference.

### STATEMENT OF GOVERNMENT INTEREST

This invention was made with government support under contract number W911NF17C0008 awarded by the U.S. Defense Advanced Research Projects Agency (DARPA), and grant number 1R43AT00952201 awarded by the U.S. National Institutes of Health (NIH). The government has certain rights in the invention.

### SEQUENCE LISTING

The XML file submitted herewith via Patent Center, named “TIER-003-02US.xml” created on Oct. 17, 2023, having a size of 60 KB, is hereby incorporated by reference in its entirety.

### FIELD

The disclosure relates to cell-free compositions and use thereof, particularly in the biodiscovery of natural products.

### BACKGROUND

There is extensive biological data encoded in DNA that is of unknown function. This data varies from naturally derived proteins, peptides, and molecular control components to semi-synthetic and engineered variants thereof. With the advent of high-throughput sequencing, as of 2017, more than 2.7 trillion bases of information are known, and only a small fraction are expressed. Tools that are able to determine the products of this DNA will be essential to understanding this information.

Separately, synthetic biology has emerged as an important field for which essential processes can be understood and engineered. Within this field there are both natural, semi-synthetic, and engineered genes, regulatory parts, and other components that are in need of testing.

Despite efforts and progresses, current approaches are limited to conduct high-throughput functional genomics to determine products from DNA and to promote synthetic biology approaches. Challenges still remain in developing engineering-driven approaches and systems to accelerate the design-build-test cycles required for reprogramming existing biological systems, constructing new biological systems and testing genetic circuits for transformative future applications in diverse areas including biology, engineering, green chemistry, agriculture and medicine.

An in vitro transcription-translation cell-free system (Shin & Noireaux, 2012; Sun et al., 2013) has been developed which allows for the rapid prototyping of genetic constructs (Sun, Yeung, Hayes, Noireaux, & Murray, 2014) in an environment that behaves similarly to a cell (Niederholtmeyer, Sun, Hori, & Yeung, 2015; Takahashi et al., 2015). One of the main purposes of working in vitro is to be able to generate fast speeds—in vitro, reactions can take 8 hours and can scale to thousands of reactions a day, a multi-fold improvement over similar reactions in cells (Sun et al., 2014). Despite the potential of this cell-free system, it needs be fine-tuned when used in different applications to achieve optimal results.

Natural products have played key roles over the past century in advancing our understanding of biology and in the development of medicine. Research in the 20th century identified many classes of natural products with four groups being particularly prevalent: terpenoids, alkaloids, polyketides, and non-ribosomal peptides. The genome sequencing efforts of the first decade of the 21st century have revealed that another major class is formed by ribosomally synthesized and post-translationally modified peptides. These molecules are produced in all three domains of life, their biosynthetic genes are ubiquitous in the currently sequenced genomes and transcriptomes, and their structural diversity is vast. The extensive post-translational/co-translational modifications endow these peptides with structures not directly accessible for natural ribosomal peptides, typically restricting conformational flexibility to allow better target recognition, to increase metabolic and chemical stability, and to augment chemical functionality.

Thus, a need exists for tools that allow the rapid, efficient discovery of national products, such as the cell-free systems disclosed herein.

### SUMMARY

In one aspect, provided herein is a composition for in vitro transcription and translation, comprising:
a) a treated cell lysate derived from one or more organisms such as bacteria, archaea, plant or animal;
b) a plurality of supplements for gene expression;
c) an energy recycling system for providing adenosine triphosphate and recycling adenosine diphosphate; and
d) an engineered propeptide operably linked to a stabilizing domain.

In some embodiments, the cell lysate is substantially free of protease.

The plurality of supplements can include reagents for transcription and translation, and optionally can include one or more non-canonical amino acids.

The stabilizing domain in some embodiments is linked to the propeptide via a linker, preferably a peptide linker comprising Gly and Ser, more preferably Gly-Gly-Gly-Gly-Ser-Ser (SEQ ID NO.: 22), Gly-Gly-Ser-Gly (SEQ ID NO.: 23), or Gly-Gly-Ser-Gly-Gly-Gly-Gly-Ser-Gly-Gly (SEQ ID NO.: 24).

In some embodiments, the engineered propeptide contains one or more protease sites that allow the stabilizing domain to be cleaved away, such as Tobacco Etch Virus (TEV) sites, PreScission Protease sites, Thrombin Protease sites, Factor Xa protease sites, and Enterokinase protease sites; and wherein the engineered propeptide contains a tag for detection by small molecule interactions, antibodies, affinity purification, or other reagents, such as FLASH/REASH sites, MBP, NusA, GST, His6, CBP, FLAG, HA, HBH, Myc, S-tag, SUMO, TAP, TRX, V5.

In some embodiments, the engineered propeptide is separately synthesized and added exogenously to the composition. In certain embodiments, the engineered propeptide contains a modification so as to resist proteolysis, preferably one or more non-canonical amino acids or a post-translation modification of an existing amino acid, or a stapled peptide.

In some embodiments, the composition can further include an engineered nucleic acid, such as DNA and/or mRNA, designed to express the engineered propeptide in the composition.

In various embodiments, the engineered propeptide can be provided from a variant library. The variant library can be pre-designed to include a plurality of propeptide variants, each having one or more mutations. The mutations can be randomized amino acid mutations, or targeted mutations designed to introduce a desired change in function, activity, stability, etc.

The composition can, in some embodiments, further include an unstructured peptide provided at no less than 0.1 mg/ml concentration in the composition.

In various embodiments, the composition is designed to produce a natural product, preferably a ribosomal natural product, more preferably a amatoxin, phallotoxin, bottromycin, cyanobactin, lanthipeptide, lasso peptide, linear azol(in)e-containing peptide, microcin, thiopeptide, autoinducing peptide, bacterial head-to-tail cycized peptide, conopeptide, cyclotide, glyocin, linearidin, microviridin, orbitide, proteusin, sactipeptide, toxin, or venom.

The composition may comprise one or more enzymes for modifying the natural product to produce a modified variant thereof. At least a portion of the one or more enzymes can be provided in the cell lysate. The composition can additionally or alternatively further comprise an engineered genetic circuit designed to express at least a portion of the one or more enzymes.

In some embodiments, the natural product can be further modified outside of the composition to produce a modified variant thereof.

The composition in some embodiments, is engineered to produce an antibiotic, herbicide, pesticide, insecticide, animal feed additive, signaling molecule, receptor agonist, receptor antagonist, activator, inhibitor, quorum sensing molecule, or anticancer therapeutic, toxin, or venom.

In some embodiments, the engineered nucleic acid or engineered genetic circuit can be derived from a microbiome, preferably human gut, animal, oral, skin, vaginal, soil, ocean, rhizosome, umbilical, vaginal, conjunctival, intestinal, stomach, nasal, gastrointestinal tract, or urogenital tract microbiomes.

The composition in some embodiments, can further include a crowding agent, preferably present at no less than 0.1% (w/v), wherein more preferably the crowding agent is polyethylene glycol present at no greater than 0.2% (w/v).

Also provided herein is a method of synthesizing a propeptide in vitro, comprising:
a) providing any one of the compositions disclosed herein; and
b) expressing the engineered propeptide in the composition.

Another aspect relates to a method of preparing a composition for in vitro transcription and translation, comprising:
a) providing a composition comprising:
   a treated cell lysate derived from one or more organisms such as bacteria, archaea, plant or animal;
   a plurality of supplements for gene expression; and
   an energy recycling system for providing adenosine triphosphate and recycling adenosine diphosphate;
b) determining that the composition is substantially free of proteases;
c) providing an engineered nucleic acid, such as DNA and/or mRNA, designed to encode a propeptide; and
d) expressing the propeptide in the composition.

In some embodiments, the composition is substantially free of proteases due to the presence of an unstructured peptide at no less than 0.1 mg/ml concentration to competitively deplete proteases, or due to genetic engineering of the organisms to remove proteases either directly or through application of tags against which to remove proteases during lysate production, or due to presence of reagents that specifically or non-specifically targets proteases. The cell lysate can be derived from any cells, such as is Rhodococcus jostii, Vibrio natriegens, Clostridium acetobutylicum, or HeLa cells.

In certain embodiments, the determining step comprises mixing the composition with an effective amount (e.g., 1 ug) of a test, unstructured peptide and determining that at least 10% of the test peptide remains after incubation for about 60 minutes.

### BRIEF DESCRIPTION OF THE DRAWINGS

FIG. 1 provides an overview of cell-free expression. In cell-free expression, a host is converted into a lysate and supplied with factors to enable the conversion of DNA to mRNA and protein.

FIG. 2 provides a comparison of traditional heterologous expression to cell-free expression.

FIG. 3 shows Conditions of different Vibrio natrigens cell-free systems, where each version of Vibrio natrigens cell-free systems is based on Sun et al. (2013), but with select modifications as listed.

FIG. 4 plots behavior of different Vibrio natrigens cell-free reactions, compared to productivity of E. coli cell free reactions and Streptomyces lividans cell-free reactions.

FIG. 5 shows toxicity of expression of sample protease inhibitors to a E. coli cell-free reaction. Cell-free systems were produced according to Sun et al. (2013), and the expression of a saturating amount of a positive control plasmid producing GFP (40019, 21p) is measured after 8 hours as a function of working concentration of protease inhibitor added per manufacturer instructions, in uM.

FIG. 6 outlines a microcin J25 expression strategy. We express microcin J25 in cell-free by assembling DNA of mcjA, mcjB, and mcjC onto T7 promoters, and run a 384-sample experiment varying the concentration of each DNA. Pooled samples can then be quickly screened to ensure correct structure. The samples can be heated to 121 C for 10 min, extracted with methanol, and assayed by HPLC (to determine size) and by MALDI/TOF or HPLC-MS/MS (to determine if lariat knot structure is preserved). Since microcin J25 is heat-stable, a heat step can rapidly determine proper folding.

FIG. 7 shows microcin J25 kills E. coli and is produced in cell-free systems. (A) Cell-free assay of mccj25 activity. Coexpression of GFP with only the complete microcin J25 cluster (mcjA-C) knocks down GFP expression. Substituting with non-microbicidal caluosegnin A or incomplete sets have no effect on GFP expression. (B) Inhibition of E. coli in vivo growth by purified microcin J25. Data, final concentrations.

FIG. 8 shows microcin J25 kills E. coli and is produced in cell-free systems and can be visualized through a high-throughput experiment.

FIG. 9 demonstrates high-throughput methods of screening known lassos that are active against E. coli RNA polymerase and other novel lassos can identify other functional lasso products.

FIG. 10 shows detection of cyclized microcin J25, acinetodin, klebsidin and a predicted cluster SVB-BGC-7 (calculated most abundant isotopes [M+2H]²⁺ 1054.519, 790.819, 1017.466, and 1292.657 respectively) from a cell-free reaction on a LC-MS qTOF. All masses within <10 ppm error.

FIG. 11 plots detection of klebsidin (calculated [M+H]⁺ 2033.918) from a cell-free reaction on a MALDI matches modelled ion distribution with <6 ppm error.

FIG. 12 shows PURExpress detection of tagged vs. untagged versions of lazA propeptide. SDS Page 4-12% Tris plus gel of non-tagged lazA (992) vs 5′cat-tagged-lazA (1071) expressed in PURExpress at equimolar concentration after 2 hours. Also run is cat-tagged and untagged version of McjA, and a propeptide from a metagenomic dataset (BDBN01000087.1A_est).

FIG. 13 describes a pipeline for novel natural product detection from bioinformatics to assembly to expression (in single format or high-throughput format) to detection to functional assays.

FIG. 14 shows degradation of predicted ribosomal propeptide in PURExpress™. Shown is a SDS-PAGE gel, visualized with Comassie Blue stain, of the expression of predicted propeptide 981 in either a PURExpress™ reaction (1:0, NA), or a PUREexpress™ reaction mixed with varying concentrations of E. coli cell-free systems (10%, 1:10, or 20%, 1:5) and incubated at 29 C for 0 min, 5 min, or 60 min.

FIG. 15 shows degradation of predicted ribosomal propeptide in varied lysate systems. Shown is a SDS-PAGE gel, visualized with Comassie Blue stain, of the expression of predicted propeptide 981 in either a PURExpress™ reaction (PURExpress only), or a PUREexpress™ reaction mixed with 10% of varied non-E. coli cell-free systems and not incubated or incubated for 1 hr. at 29 C. BY2, Tobacco BY2; WCE, whole cell extract.

FIG. 16 shows lack of degradation of predicted ribosomal propeptide in varied E. coli and V. natrigens cell-free systems. Shown is a SDS-PAGE gel, visualized with Comassie Blue stain, of predicted propeptide 981 expressed in a PURExpress™ reaction and incubated for an hour in a 5:1 volume ratio with E. coli extract or V. natriegens extracts of varying protein synthesis capability to visualize the extent of propeptide degradation. Fairly equivalent amounts of propeptide remained after incubation with the most active V. natriegens extract (eVN2.2) as after incubation with unoptimized V. natriegens extract (eVN1).

FIG. 17 shows detection of linearized core vs. cyclized core from cell-free reactions with microcin J25 (mcj25) and klebsidin. Significant differences in enzyme efficiency were observed between clusters. Cell free reactions were expressed overnight with 4 nM of each component gene and frozen at −80° C. These 180 μl reactions were later thawed, treated with 0.5% FA, extracted twice with 2:1 volumes of n-butanol. Organic phase was removed and dried, then resuspended in 25 μl of water. Peaks were detected on and agilent 6510-QTOF as they eluted from a zorbax-300SB C18 column in a reverse phase gradient of 26-98% acetonitrile.

FIG. 18 shows expression of MBP-A fusion vs. MBP only in cell-free systems. Linear constructs were expressed in 10 μl cell free reactions with 2% flurotect (a tRNA carrying a BODIPY labelled lysine) and 8 nM DNA constructs for MBP-A or MBP (1065, 1066). 41 of these reactions were prepared in 241 sample buffer (50 mM bis-Tris, 2% SDS, 10% glycerol) and run on a 4-12% bis-tris PAGE gel (ThermoFisher Scientific) at 200V for 20 min.

FIG. 19 shows comparison of expression with and without crowding agent in E. coli cell-free systems. E. coli cell-free systems are prepared according to Sun et al. (2013) JoVE, but the crowding agent is substituted out for different percentages (w/v) of PEG and Ficoll 400. Shown is expression of 1 nM 21p (40019) after 8 hours, plotted as uM endpoint and maximum rate of expression.

FIG. 20 shows expression using native or T7 transcription machinery in V. natriegens extract with varying concentrations of crowding agents. 8 nM of GFP reporter constructs that rely on either native transcription machinery (21p) or the same plasmid with the T7 promoter were used to assess the degree to which macromolecular crowding affects protein expression in V. natriegens extract.

While the above-identified drawings set forth presently disclosed embodiments, other embodiments are also contemplated, as noted in the discussion. This disclosure presents illustrative embodiments by way of representation and not limitation. Numerous other modifications and embodiments can be devised by those skilled in the art which fall within the scope and spirit of the principles of the presently disclosed embodiments.

### DETAILED DESCRIPTION

Disclosed herein, in one aspect, is a cell-free system that can be used to explore natural products and their variants that result from metagenomic as well as synthetic sources. One reservoir of natural products are actinomycetes, a group of gram-positive bacteria, typically with high genomic G+C content, that are a source of diverse bioactive secondary metabolites. Of all antibiotics known, 66% are produced by actinomycetes. Representative marketed natural products include streptomycin, erythromycin, clauvanic acid, chloramphenicol, and amorpha-1,4-diene. Two members of the group, Streptomycetales and Pseudonocardiales, were found to have a per-genome average of ˜20 secondary metabolites (defined as PKS Types I and II, NRPS, lanthipeptides, thiazole-oxazole modified microcins, and NRPS-independent siderophores). Currently characterized metabolites represent only 10% of bioinformatically detected secondary metabolites, indicating a large unexplored database. Compounding the difficulty of secondary metabolite detection is low native production rates—for example, daptomycin (a lipopeptide antibiotic) was identified after fermentation of 10⁷ strains. By removing the hurdle of producing normally silent secondary metabolites in cells, one can remove native negative regulatory methods and the costs and time to conduct experiments in vivo.

The compositions and methods disclosed herein relieve the hurdle of expressing natural products and synthetic variants thereof by utilizing cell-free expression system in lieu of cellular expression. This allows for the expression of otherwise silent natural products, or natural product variants that cannot be catalyzed by traditional heterologous expression or by isolating natural products. The focus of the compositions and methods proposed is on expressing ribosomal natural products and synthetic variants. Techniques to express ribosomal natural products, including the stabilization of propeptide/prepeptide starting precursors, are disclosed herein.

### Definitions

For convenience, certain terms employed in the specification, examples, and appended claims are collected here. Unless defined otherwise, all technical and scientific terms used herein have the same meaning as commonly understood by one of ordinary skill in the art to which this disclosure belongs.

The articles “a” and “an” are used herein to refer to one or to more than one (i.e., at least one) of the grammatical object of the article. By way of example, “an element” means one element or more than one element.

As used herein, the term “about” means within 20%, more preferably within 10% and most preferably within 5%. The term “substantially” means more than 50%, preferably more than 80%, and most preferably more than 90% or 95%.

As used herein, “a plurality of” means more than 1, e.g., 2, 3, 4, 5, 6, 7, 8, 9, 10, 11, 12, 13, 14, 15, 16, 17, 18, 19, 20, or more, e.g., 25, 30, 40, 50, 60, 70, 80, 90, 100, 200, 300, 400, 500, or more, or any integer therebetween.

The term “natural product” refers to biological products that can be found in nature. The compositions and systems disclosed herein can be used as effective tools to discover unknown natural products. In some embodiments, the natural product can be a ribosomal natural product (also referred to as a Ribosomally synthesized and post translationally modified peptide or RiPP). Examples include but are not limited to, amatoxin, phallotoxin, bottromycin, cyanobactin, lanthipeptide, lasso peptide, linear azol(in)e-containing peptide, microcin, thiopeptide, autoinducing peptide, bacterial head-to-tail cycized peptide, conopeptide, cyclotide, glyocin, linearidin, microviridin, orbitide, proteusin, sactipeptide, toxin, and/or venom.

The term “modified variant” of a natural product refers to a non-naturally existing product that is a variant of the natural product. Such variant can contain one or more modifications such as non-canonical amino acids, post-translational modifications, methylation, glycosylation, prenylation, dehydration, cyclodehydration, macrocyclization, thioester linkages, crosslinks, chelation, thioamide linkages, heterocycles. Such variants may also be rationally engineered by utilizing known information about the production of a natural product and varying components that are not necessary for the natural product processing. Examples include scaffolding of propeptide regions for processing by native machinery to produce variants of natural products capable of binding receptors, or providing modified inputs (eg. decorated side chains) that are then processed to produce modified natural products with unique properties. Modified variants of natural products can have favorable properties beyond the original natural product (e.g., cancer therapeutic, anti-proteolysis, receptor binding, increased specificity, etc.). The production of modified variants is similar to the process of diversifying scaffolds in synthetic chemistry, but utilizes biological techniques.

As used herein, the terms “nucleic acid,” “nucleic acid molecule” and “polynucleotide” may be used interchangeably and include both single-stranded (ss) and double-stranded (ds) RNA, DNA and RNA:DNA hybrids. These terms are intended to include, but are not limited to, a polymeric form of nucleotides that may have various lengths, including deoxyribonucleotides and/or ribonucleotides, or analogs or modifications thereof. A nucleic acid molecule may encode a full-length polypeptide or RNA or a fragment of any length thereof, or may be non-coding.

Nucleic acids can be naturally-occurring or synthetic polymeric forms of nucleotides. The nucleic acid molecules of the present disclosure may be formed from naturally-occurring nucleotides, for example forming deoxyribonucleic acid (DNA) or ribonucleic acid (RNA) molecules. Alternatively, the naturally-occurring oligonucleotides may include structural modifications to alter their properties, such as in peptide nucleic acids (PNA) or in locked nucleic acids (LNA). The terms should be understood to include equivalents, analogs of either RNA or DNA made from nucleotide analogs and as applicable to the embodiment being described, single-stranded or double-stranded polynucleotides. Nucleotides useful in the disclosure include, for example, naturally-occurring nucleotides (for example, ribonucleotides or deoxyribonucleotides), or natural or synthetic modifications of nucleotides, or artificial bases. Modifications can also include phosphorothioated bases for increased stability.

As used herein, unless otherwise stated, the term “transcription” refers to the synthesis of RNA from a DNA template; the term “translation” refers to the synthesis of a polypeptide from an mRNA template. Translation in general is regulated by the sequence and structure of the 5′ untranslated region (5′-UTR) of the mRNA transcript. One regulatory sequence is the ribosome binding site (RBS), which promotes efficient and accurate translation of mRNA. The prokaryotic RBS is the Shine-Dalgarno sequence, a purine-rich sequence of 5′-UTR that is complementary to the UCCU core sequence of the 3′-end of 16S rRNA (located within the 30S small ribosomal subunit). Various Shine-Dalgarno sequences have been found in prokaryotic mRNAs and generally lie about 10 nucleotides upstream from the AUG start codon. Activity of a RBS can be influenced by the length and nucleotide composition of the spacer separating the RBS and the initiator AUG. In eukaryotes, the Kozak sequence lies within a short 5′ untranslated region and directs translation of mRNA. An mRNA lacking the Kozak consensus sequence may also be translated efficiently in an in vitro system if it possesses a moderately long 5′-UTR that lacks stable secondary structure. While E. coli ribosome preferentially recognizes the Shine-Dalgarno sequence, eukaryotic ribosomes (such as those found in retic lysate) can efficiently use either the Shine-Dalgarno or the Kozak ribosomal binding sites.

As used herein, the term “host” or “host cell” refers to any prokaryotic or eukaryotic single cell (e.g., yeast, bacterial, archaeal, etc.) cell or organism. The host cell can be a recipient of a replicable expression vector, cloning vector or any heterologous nucleic acid molecule. Host cells may be prokaryotic cells such as species of the genus Escherichia or Lactobacillus, or eukaryotic single cell organism such as yeast. The heterologous nucleic acid molecule may contain, but is not limited to, a sequence of interest, a transcriptional regulatory sequence (such as a promoter, enhancer, repressor, and the like) and/or an origin of replication. As used herein, the terms “host,” “host cell,” “recombinant host” and “recombinant host cell” may be used interchangeably. For examples of such hosts, see Green & Sambrook, 2012, Molecular Cloning: A laboratory manual, 4th ed., Cold Spring Harbor Laboratory Press, New York, incorporated herein by reference.

One or more nucleic acid sequences can be targeted for delivery to target prokaryotic or eukaryotic cells via conventional transformation techniques. As used herein, the term “transformation” is intended to refer to a variety of art-recognized techniques for introducing an exogenous nucleic acid sequence (e.g., DNA) into a target cell, including calcium phosphate or calcium chloride co-precipitation, conjugation, electroporation, sonoporation, optoporation, injection and the like. Suitable transformation media include, but are not limited to, water, CaCl₂), cationic polymers, lipids, and the like. Suitable materials and methods for transforming target cells can be found in Green & Sambrook, 2012, Molecular Cloning: A laboratory manual, 4th ed, Cold Spring Harbor Laboratory Press, New York, incorporated herein by reference, and other laboratory manuals.

As used herein, the term “selectable marker” or “reporter” refers to a gene, operon, or protein that upon expression in a host cell or organism, can confer certain characteristics that can be relatively easily selected, identified and/or measured. Reporter genes are often used as an indication of whether a certain gene has been introduced into or expressed in the host cell or organism. Examples, without limitation, of commonly used reporters include: antibiotic resistance (“abR”) genes, fluorescent proteins, auxotropic selection modules, β-galactosidase (encoded by the bacterial gene lacZ), luciferase (from lightning bugs), chloramphenicol acetyltransferase (CAT; from bacteria), GUS (β-glucuronidase; commonly used in plants) green fluorescent protein (GFP; from jelly fish), and red fluorescent protein (RFP). Typically host cells expressing the selectable marker are protected from a selective agent that is toxic or inhibitory to cell growth.

The term “engineer,” “engineering” or “engineered,” as used herein, refers to genetic manipulation or modification of biomolecules such as DNA, RNA and/or protein, or like technique commonly known in the biotechnology art.

A “circuit” or “genetic circuit” as used herein refers to a collection of parts (e.g., genes or other genetic elements) that undergo transcription and/or translation to produce mRNA or proteins, respectively (each an “output” of the part). The part output can interact with other parts (for example to regulate transcription or translation) or can interact with other molecules in the cell-free system (e.g., small molecules, DNA, RNA or propeptides). For example, a circuit can be a metabolic pathway or a genetic cascade, which can be naturally occurring or non-naturally occurring, artificially engineered. Each part in the circuit can include a set of components or genetic modules, e.g., a promoter, ribosome binding site (RBS), coding sequence (CDS) and/or terminator. These components may be interconnected or assembled in different ways to implement different parts, and the resultant parts may be combined in different ways to create different circuits or pathways. In addition to these parts, the circuit may contain additional molecular species that are present in a cell or in the cell's environment that the components interact with. In one example, a genetic circuit can be designed to express one or more enzymes that modify the propeptides.

As described herein, “genetic module” and “genetic element” may be used interchangeably and refer to any coding and/or non-coding nucleic acid sequence. Genetic modules may be operons, genes, gene fragments, promoters, exons, introns, regulatory sequences, tags, or any combination thereof. In some embodiments, a genetic module refers to one or more of coding sequence, promoter, terminator, untranslated region, ribosome binding site, polyadenlylation tail, leader, signal sequence, vector and any combination of the foregoing. In certain embodiments, a genetic module can be a transcription unit as defined herein.

As used herein, the term “operably linked” means a first genetic element (e.g., propeptide encoding DNA) is engineered to be in the same nucleic acid molecule, and is in a functional relationship, with a second genetic element (e.g., a stabilizing domain encoding DNA) such that both can be, e.g., expressed as intended.

Other terms used in the fields of recombinant nucleic acid technology and molecular and cell biology as used herein will be generally understood by one of ordinary skill in the applicable arts.

### Composition of In Vitro Transcription and Translation

The in vitro transcription and translation system is a system that is able to conduct transcription and translation outside of the context of a cell. In some embodiments, this system is also referred to as “cell-free system”, “cell-free transcription and translation”, “TX-TL”, “lysate systems”, “in vitro system”, “ITT”, or “artificial cells.” In vitro transcription and translation systems can be either purified protein systems, that are not made from hosts, as referenced by (Shimizu et al., 2001), or can be made from a host strain that is formed as a “lysate.” Those skilled in the art will recognize that an in vitro transcription and translation requires transcription and translation to occur, and therefore does not encompass reactions with purified enzymes.

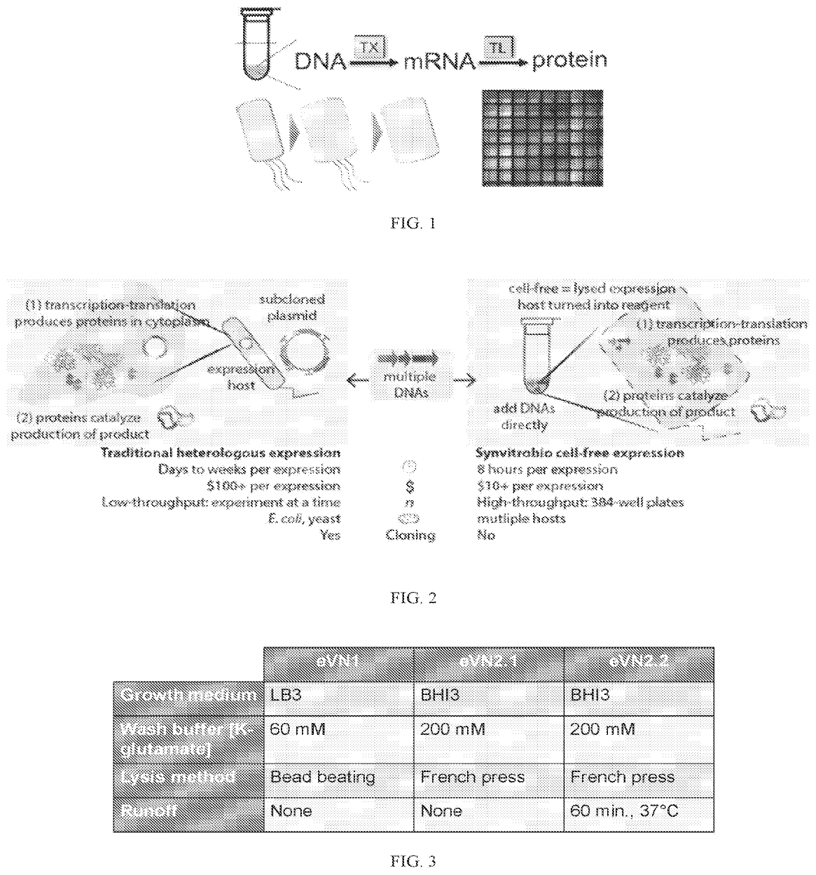

Cell-free transcription-translation is described in FIG. 1. Top, cell-free expression that takes in DNA and produces protein that catalyzes reactions. Bottom, diagram of cell-free production and representative data collected in 384-well plate format of GFP expression. Cell-free approaches contrasted to cellular approaches are described in FIG. 2. Cell-free platform allows for protein expression from multiple genes without live cells. Cell-free production biotechnology methods produce lysates from prokaryotic cells that are able to take recombinant DNA as input and conduct coupled transcription and translation to output enzymatically active protein. Cell-free systems take only 8 hours to express, rather than days to weeks in cells, since there is no need for cloning and transformation. They are also at least 10-fold cheaper to run than cells, and can be run in high-throughput as reactions are the equivalent of a reagent and used in a 384-well plate. Typical yields of prokaryotic systems are 750 μg/mL of GFP (30 μM). Extracts multiple cell-free systems can be implemented, conducted at scales from 10 μl up to 10 mL.

Directions on how to make the lysate component of cell-free systems, particularly from E. coli, can be found in (Sun et al., 2013), which is incorporated herein by reference in its entirety. While this procedure is adapted for E. coli cell-free systems, it can be used to produce other cell-free systems from other organisms and hosts (prokaryotic, eukaryotic, archaea, fungal, etc.) Examples, without limitation, of the production of other cell-free systems include Streptomyces spp. (Thompson, Rae, & Cundliffe, 1984), Bacillus spp. (Kelwick, Webb, MacDonald, & Freemont, 2016), and Tobacco BY2 (Buntru, Vogel, Spiegel, & Schillberg, 2014), where directions are incorporated herein by reference in its entirety. The process for producing lysates in this disclosure involves growing a host in a rich media to mid-log phase, followed by washes, lysis by French Press and/or Bead Beating Homogenization, and clarification. A lysate that has been processed as such can be referred to as a “lysate”, a “treated cell lysate”, or an “extract”.

The extract can be made from one host, multiple hosts, or mixes of multiple hosts. It is obvious to those that are skilled in the art that mixing extracts of multiple hosts, as described in U.S. Pat. No. 9,469,861 for producing carbapenems in cell-free systems and incorporated herein by reference in its entirety, may be necessary to supply cofactors that are necessary to produce different natural products.

A plurality of supplements is supplied along-side an extract to maintain gene expression. This includes necessary items for transcription and translation, such as amino acids, nucleotides, salts (Magnesium and Potassium), and buffers. A review of supplements can be found in (Chiao, Murray, & Sun, 2016), incorporated herein by reference in its entirety. This can also include optional items that assist transcription and translation, such as phage polymerases, T7 RNA polymerase, SP6 phage polymerase, cofactors, elongation factors, nanodiscs, vesicles, and antifoaming agents.

An energy recycling system is necessary to drive synthesis of mRNA and proteins by providing ATP (adenosine triphosphate) to a system and by maintaining system homoeostasis by recycling ADP (adenosine diphosphate) to ATP, by maintaining pH, and generally supporting a system for transcription and translation. A review of energy recycling systems can be found in (Chiao et al., 2016), incorporated herein by reference in its entirety. Examples, without limitation, of energy recycling systems that can be used include 3-PGA (Sun et al., 2013), PANOx (D.-M. Kim & Swartz, 2001), and Cytomim (Jewett & Swartz, 2004).

A polypeptide under 110 amino acids is supplied to the composition. A polypeptide of this size can also be referred to as a “prepeptide”, “propeptide”, “prepropeptide”, “structural peptide”, “pro-region”, “intervening region”, “precursor peptide”, or “leader peptide.” This polypeptide, typically between 20-110 residues, is characteristic of ribosomally-synthesized and post-translationally-modified peptides (RiPPs), or ribosomal natural product, as described in (Arnison et al., 2013), incorporated herein by reference in its entirety. In some embodiments, the polypeptide does not have significant tertiary structure and is therefore prone to proteolysis.

In some embodiments, a DNA is supplied that that can produce the polypeptide by utilizing transcription and translation machinery in the lysate and/or additions to the lysate. This DNA has regulatory regions, such as under the OR2-OR1-Pr promoter (Sun et al., 2014) the T7 promoter or T7-lacO promoter, along with a RBS region, such as the UTR1 from lambda phage (Sun et al., 2014), or BCD units (Mutalik et al., 2013). The DNA can be linear or plasmid. An example of a sequence is provided in SEQ ID NO.: 1.

In other embodiments, a mRNA is supplied that utilizes translational components in the lysate and/or additions to the lysate to produce the polypeptide. This mRNA can be from a purified natural source, or from a synthetically generated source, or can be generated in vitro, e.g., from an in-vitro transcription kit such as HiScribe™, MAXIscript™, MEGAscript™, mMESSAGE MACHINE™ MEGAshortscript™.

In other embodiments, the polypeptide is directly supplied. This polypeptide can be from a purified natural source, a synthetically generated source (custom peptide synthesis), or from another in vitro transcription and translation kit. In some embodiments, the polypeptide is directly supplied to introduce non-canonical or non-natural amino acids at high yield (Hong, Kwon, & Jewett, 2014). In other embodiments, the polypeptide is directly supplied to introduce non-naturally occurring polypeptides for applications in scaffolding for drug or receptor binding design, as described in (T. A. Knappe et al., 2011) and incorporated herein by reference in its entirety. Those skilled in the art will recognize that the polypeptide will need to be relatively devoid of contaminants, such as salts, to not interfere with the in vitro transcription and translation reaction.

The polypeptide under 110 amino acids is modified in the reaction by endogenous or exogenous factor to produce a product. In some embodiments, the factor is endogenous to the in vitro transcription and translation system, such as if the factor is supplied by the lysate. An exemplary example is provided for lariatin in (Inokoshi, Matsuhama, Miyake, Ikeda, & Tomoda, 2012; Iwatsuki, Uchida, & Takakusagi, 2007), incorporated herein by reference in its entirety, where the correct production of laraitin requires the presence of the R. jostii K01-B0171 strain and cannot be produced by heterologous expression in a E. coli strain, thereby implying that a factor endogenous to a lysate from R. jostii K01-B0171 would be required.

In some embodiments, the factor is exogenous to the in vitro transcription and translation system. Examples, without limitation, are ribosomal natural product pathways, such as those that produce microcin J25, klebsidin, and lactazole, as provided in examples herein, where factors necessary to modify the supplied polypeptide under 110 amino acids can be supplied as DNA, mRNA, or protein. In these cases, the factor may be part of the natural product biosynthetic machinery (e.g., (cyclizing enzyme, cleaving enzyme), or may conduct modifications on the supplied polypeptide under 110 amino acids or a variant (e.g., decorating enzyme, dehydration reactions, Michael-type additions). A review of potential factors are found in (Ortega & van der Donk, 2016), incorporated herein by reference in its entirety. In certain embodiments, the cell-free transcription and translation reactions, as described in WO2016134069A1, Niederholtmeyer et al., 2015, and Sun et al., 2014, each incorporated herein by reference in its entirety, can be utilized.

Unique to this application is the inclusion of a polypeptide of size under 110 amino acids that is then modified within the cell-free reaction. While polypeptides have been included into cell-free reactions, these polypeptides typically do not physically modify a externally supplied polypeptide. In cases were the polypeptide does modify an externally supplied polypeptide, the polypeptide is usually of larger size, such as an antibody or an established protein.

In some embodiments, the composition produces a natural product, or a ribosomal natural product, or “RiPP.” A description of the class is incorporated herein by reference in its entirety in (Arnison et al., 2013; Ortega & van der Donk, 2016).

In some embodiments, the composition produces a product that can be further modified to produce a non-naturally occurring Natural Product. This may be done through adding modifying enzymes, either directly or through DNA or mRNA that is translated/transcribed to produce the modifying enzyme, that do not naturally modify the intended product, but are promiscuous enough to modify the supplied product, as described in (“Modularity of RiPP Enzymes Enables Designed Synthesis of Decorated Peptides,” 2015), incorporated herein by reference in its entirety. This may also be done external to the cell-free reaction.

In some embodiments, non-canonical amino acids are utilized in the composition. Non-canonical amino acids can may be found naturally in the cellular-produced product, or can be artificially added to the product to produce desirable properties, such as tagging, visualization, resistance to degradation, or targeting. While implementation of non-canonical amino acids is difficult in cells, in cell-free systems implementation rates are higher due to the ability to saturate with the non-canonical amino acid. Examples, without limitation, of non-canonical amino acids, including ornithine, norleucine, homoarginine, tryptophan analogs, biphenylalanine, hydrolysine, pyrrolysine, or as described in (Blaskovich, 2016) broadly for medicinal chemistry and specifically in (Baumann et al., 2017) for natural products, are incorporated herein by reference in its entirety.

In some embodiments, the input polypeptides and/or factors are derived from environmental sequences or nucleic acids. These nucleic acids can be further derived from microbiomes, such as human gut, animal, oral, skin, vaginal, soil, ocean, rhizosome, umbilical, vaginal, conjunctival, intestinal, stomach, nasal, gastrointestinal tract, or urogenital tract. Of particular interest is the human gut microbiome, which is known to be highly overexpressed in ribosomal natural products, and the soil microbiome, from which many commercially valuable natural products (such as ‘nisin’) have been isolated. Those skilled in the art will recognize that the composition can produce the desired product using these environmental sequences and effectively emulate the activity of the host cell by doing so, thereby acting as an “artificial cell” or an alternate heterologous expression platform. In other embodiments, the input polypeptides and/or factors are derived from non-environmental (synthetic) sources. This can be to produce non-natural analogs of natural products or to speed up production of natural products (e.g., modifying flexible residues of a input lasso peptide propeptide to produce a scaffold that has the bioactivity characteristic of an antibody but the ability to enter cells, enzyme evolution to accelerate activity of limiting enzymes, enzyme evolution to allow the production of new products).

In some embodiments, the product of the reaction is a molecule that has bio-active activity. Those skilled in the art will recognize that if the input sequences are environmental, they are likely to be evolved by nature to product useful, bio-active molecules. The activity of the bio-active molecule can include antibiotic, herbicide, pesticide, insecticide, animal feed additive, signaling molecule, receptor agonist, receptor antagonist, activator, inhibitor, quorum sensing molecule, or anticancer therapeutic, toxin, or venom. In other embodiments, the bio-active molecule is derived from synthetic input sources. In these cases, many times knowledge of a known bio-activity is utilized to produce the synthetic variant, for example computationally designing a structure to bind a receptor, NMR, or crystallographiclly-defined site, and then scaffolding a polypeptide and utilize the composition and known natural product chemistry to produce a agonist/antagonist/effector.

In some embodiments, crowding agents are used in the reaction to simulate the macromolecular crowding activity in the cell and to encourage the protein-protein and protein-nucleic acids interactions necessary to drive the reaction to completion. Macromolecular crowding is an important effect in biochemical reactions, affecting, transcription, DNA replication, and protein folding. Macromolecular crowding helps to stabilize proteins in their folded state by varying excluded volume—the volume inaccessible to the proteins due to their interaction with macromolecular crowding agents. This is critical to cells; for example, E. coli cytoplasm contains 300-400 mg/mL of macromolecules. Examples, without limitation, of typical crowding agents, which are typically, e.g., above 100 Daltons, above 150 Daltons, or above 200 Daltons, include: Ficoll, polyethylene glycol, polyethylene oxide, cyclodextrin, dextran, bovine serum antigen, glucose, among others. There are assumptions that (1) crowding is not critical for some cell-free systems to function, especially those that are driven by T7 expression. This is a reasonable assumption, as the interaction of T7 RNA polymerase to the T7 operator is very strong and may not need crowding conditions to occur. Also, that (2) crowding should best emulate the cellular condition, as described in the Cytomin™ system, which uses spermidine and putrescene (cations/polyamines, not crowding agents) and particularly avoids polyethylene glycol due to negative effects. However, surprisingly, in contrast to the findings of U.S. Pat. No. 8,357,529, it has been discovered herein that: (1) crowding, while not important for the interaction of T7 RNA polymerase to the T7 operator, assists the production of natural products in cell-free lysates, and (2) that polyethylene glycol is a positive effector to crowding and crowding conditions do not need to emulate cellular conditions.

Crowding can encourage the protein-protein interactions resulting from the input polypeptide of less than 110 amino acids with either endogenous and exogenous factors. While the activity of crowding agents to impact cellular expression is well-understood, there is limited work defining the activity of crowding agents with respect to cell-free systems (e.g., (Ge, Luo, & Xu, 2011; Tan, Saurabh, Bruchez, Schwartz, & LeDuc, 2013)), and no publically available work to date (other than our example demonstration) showing activity of crowding agents in encouraging either protein-protein interactions or interactions for producing ribosomal natural products.

In some embodiments, the reaction comprises more than 0.1% (w/v) of crowding agent. The crowding agent used may be from a single source, or may be a mix of different sources. The crowding agent may be from varied sizes. In some embodiments, the crowding agents used limit polyethylene glycol and its derivate, polyethylene oxide or polyoxyethylene, to less than 0.2% (w/v). While polyethylene glycol and its derivatives are similar as other crowding agents in their biochemical and biophysical effect, polyethylene glycol and its derivatives can interfere with analytical methods of downstream detection of the resulting product, which can be critical for diagnosing and/or reading out the resulting reaction. To minimize this effect, polyethylene glycol and its derivatives can be limited and substituted for other crowding agents. In addition, the size used of polyethylene glycol and its derivatives can be varied.

Protection of Peptides of Less than 110 Amino Acids in Length by Utilizing Non-Proteolytically Active Lysates

In some embodiments, peptides of less than 110 amino acids in length are easily degraded in either lysates, or in cell-free systems. Ribosomal natural products are generally produced by a propeptide that is later modified by other coding sequences to produce a final product. The propeptide, therefore, is one of the rate-limiting steps of producing a ribosomal natural product. In cells, there is proteolytic activity that can degrade unfolded, misfolded, or peptides that have no secondary or tertiary structure. However, many ribosomal natural product wildtype propeptides do not have secondary or tertiary structure, leaving them open to proteolytic degradation. In our examples, we demonstrate that in cell-free systems that have active proteolysis, the propeptide degrades away without protection. This limits the yields achievable of ribosomal natural product production in cell-free systems if no protective strategies are enforced. The protective strategies are unexpected as many of them would not be able to be applied directly to cells.

In some embodiments, to protect peptides of less than 110 amino acids in length, lysates or cell-free systems are made that are highly productive at transcription and translation but also able to avoid proteolytic activity. We demonstrate in examples that proteolytic degradation is not unique to E. coli cell-free systems, but can selectively occur in different lysates from different backgrounds. While some systems, such as E. coli cell-free systems, are enriched in proteolytic components, other systems are less enriched.

In some embodiments, the lysates and/or cell-free systems produced are made from Rhodococcus jostii, Vibrio natriegens, Clostridium acetobutylicum, HeLa whole cell extract.

In some embodiments, the lysates and/or cell-free systems produced are made from organisms that are known to be devoid of proteolytic ability due to known or predicted properties of cellular biochemistry.

In some embodiments, the lysates and/or cell-free systems that are less proteolytically active are experimentally determined. In this method, a sample test, unstructured (e.g., having no secondary or higher structures) peptide under 110 amino acids in length is provided, such as MTKRTYETPVLVSAGSFARRTGSGSPKAARDPFGRRWLP (SEQ ID NO.: 21). This unstructured peptide can be produced synthetically, or produced in a system that is devoid of proteases, such as the PURExpress™ system, where the peptide is expressed with a saturating amount of T7-encoding DNA, such as that in SEQ ID NO.: 2. The unstructured peptide and/or the solution in which the peptide is made is then combined with ideally 10%, or anywhere from 1% to 100%, of a solution of either lysates or cell-free systems derived from different hosts. This solution is incubated for different time periods at ideally 29 C, or anywhere from 20 C to 80 C, for time periods from 0 min to 60 min. The amount of peptide left at the longest time periods is compared to the amount of peptide at the shortest time periods by methods such SDS-PAGE or by selective labeling. The ratio of these numbers determine proteolytic ability. Those skilled in the art will recognize that in parallel, cell-free systems formed by those that have low proteolytic ability need to be evaluated for active transcription-translation ability.

In some embodiments, it is necessary to produce a Vibrio natriegens cell-free system that is proteolytically inactive. For eVN1, Vibrio natrigens cell-free system is produced using the methods of Sun et al. (2013), but with select modifications to the protocol outlined in FIG. 3. The Vibrio natriegens extract used in this initial screen was prepared by growing a 1 L culture of V. natriegens in LB medium with 3% NaCl at 37° C., 250 RPM, to an OD600 of 1.2 which was pelleted by centrifugation for 10,000×g for 10 minutes at 4° C. Cell pellets were washed by resuspension in a wash buffer (14 mM Mg-glutamate, 60 mM K-glutamate, 10 mM HEPES-KOH pH 7.6) followed by another spin at 10,000×g for 10 minutes. The wash step was repeated once more. The resulting pellet was resuspended in 40 ml wash buffer and transferred to a 50 mL conical tube which was spun twice at 8,000×g for 5 minutes to remove the wash buffer. The above process was conducted on ice or at 4° C. Resulting pellets were transferred to −80° C. for storage in preparation of continuation of the extract preparation protocol. On a subsequent day, the pellets were thawed on ice after addition of 0.9 mL of 4° C. wash buffer per mg pellet mass. Cells were resuspended, lysed by bead beating, and clarified according to the protocol specified in Sun et al. (2013) Journal of Visualized Experiments. The resulting extract was aliquotted and frozen in −80° C. for storage.

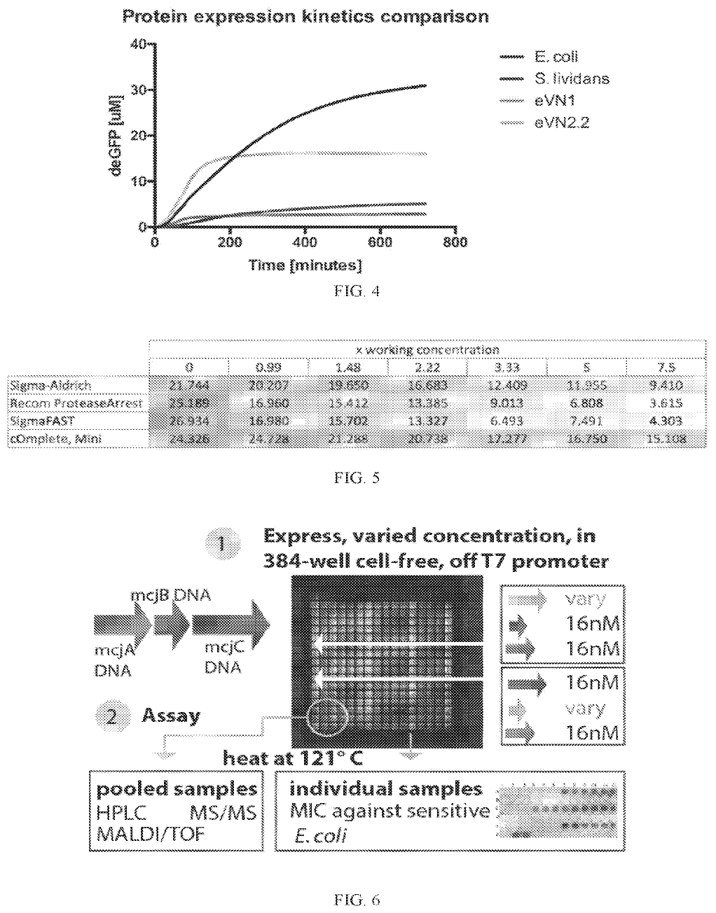

In some embodiments, modifications can be made to increase the protein synthesis capacity of the Vibrio natriegens extract expression chassis to increase transcription and translation ability. Modifications to the protocol included replacing LB with 3% NaCl with Brain Heart Infusion Broth with 3% NaCl, increasing the concentration of K-glutamate in the wash buffer to 200 mM, lysing via French press rather than bead beating, conducting a 60 minute runoff incubation of the lysate at 37° C., increasing the energy substrate and amino acid concentrations 2.5×, and increasing the volume of the extract in the final reaction to 50% of the total volume. These changes are summarized in FIG. 3. Altogether, these modifications resulted in a 5-fold increase in protein yield as measured by GFP synthesis in eVN2.2, the most productive of the Vibrio natriegens extract batches, as shown in FIG. 4.

In some embodiments, the lysates and/or cell-free systems used can be depleted of proteolytic components. The depletion can be either before or after the production the cell-free system, at either the host stage, lysate preparation stage, or post-lysate preparation stage.

In some embodiments, the depletion of proteolytic components is done by adding an effector, such as protease inhibitors. Protease inhibitors inhibit the function of proteases (enzymes that aid the breakdown of proteins). Classes of protease inhibitors include aspartic protease inhibitors, cysteine protease inhibitors, metalloprotease inhibitors, serine protease inhibitors threonine protease inhibitors, trypsin inhibitors, suicide inhibitor, transition state inhibitor, serpins, chelating agents. The inhibitors can be specific, or can be general. The inhibitors can be individual chemicals or provided in cocktail form. Examples, without limitation, of protease inhibitors include SIGMAFAST™, MS-SAFE™ cOmplete™, Halt Protease™, EDTA, pepstatin A, PMSF, E-64, bestatin, aprotinin, AEB SF, Sodium phyophosphate, beta-glycerophophate, sodium orthovandate, sodium fluoride. The method of testing protease inhibitors in cell-free systems involves providing the protease inhibitor in a cell-free system and testing the transcription-translation activity of the cell-free system through the expression of a constitutively active GFP-producing DNA (e.g., Addgene 40019 or 21p) in the presence and in the absence of the protease inhibitor, as demonstrated in FIG. 5. In parallel, the rate of proteolysis can be determined by incubating a unstructured peptide with the cell-free system with protease inhibitor added to determine amount of degradation over time. The ideal protease inhibitor is able to not suppress transcription-translation below 25% of wildtype activity while decreasing proteolysis activity. Those skilled in the art will recognize that many protease inhibitors are chelators and may reduce Mg2+ concentration and thereby effective ATP concentration in the cell-free solution. To counteract this effect, addition Mg2+ and/or ATP may need to be provided to the solution.

In some embodiments, the depletion of proteolytic components is done by adding to the solution a dummy or competing peptide that is unstructures (e.g., without any secondary or higher structures). By including dummy peptides, any present protease will competitively degrade the dummy peptide as well as the input peptide. Due to the larger amount of dummy peptides present, the input peptide can be protected from degradation. In addition, increased turnovers of protease can result in the inactivity of the protease. This has been shown for AAA+ protein degradation enzymes (e.g., ClpXP) in previous work (Sun, Kim, Singhal, & Murray, 2015) in cell-free systems. The dummy peptides will need to not contain signaling regions or other regions that can bind, recruit, activate, or inhibit proteins or molecules essential for the cell-free system to function. Any such regions will cause the dummy peptides to interfere with transcription, translation, or essential processes. These properties can be found by comparing dummy peptides to data in publically available databases (e.g., NCBI, EMBL). The ideal source of dummy peptides is generated from random amino acid sequences. The dummy peptide can be generated by peptide synthesis or by production in another cell or cell-free reaction. In a sample reaction, random dummy peptide SFAVHGIWET YLRDQMNKCP (SEQ ID NO.: 25) (or as otherwise generated by RandSeq) is synthesized, purified of contaminating components (such as salts), and resuspended in a neutral protein buffer such as 50 mM Tris-Cl, 100 mM NaCl, 1 mM DTT, 2% DMSO, pH 7.5. The dummy peptide is added to a cell-free reaction at 0.1 mg/ml, 0.5 mg/ml, 1 mg/ml, 5 mg/ml, 10 mg/ml, and the transcription-translation of the desired peptide of less than 110 amino acids is monitored. A comparison is done of adding the dummy peptide before the reaction and incubating for 60 minutes, or adding the dummy peptide concurrently with transcription-translation of the desired peptide. In addition, the toxicity of the dummy peptides is monitored by the expression of a positive control plasmid (e.g., 21p, 40019). Parallel methods for tracking ClpXP degradation of fluorescent proteins are described in (Sun et al., 2015), incorporated herein by reference in its entirety.

In some embodiments, the depletion of proteolytic components is done by genetically engineering out proteases out of a host before or during the lysate production process to effectively produce a cell-free system deprived of protease activity. However, care must be taken to not inhibit essential growth and regulatory processes of the host species while the host species is still growing. To remove these agents, host cells will be first genetically engineered using a CRISPR-Cas9, TALEN, MAGE, or other genetically engineering approach to remove proteases that do not effect growth and regulatory processes. This can be tested by conducting a cycle of genetic engineering (e.g., by inserting a nonsense codon into a putative protease) and then conducting an OD growth curve using growth conditions for producing lysate in rich media and comparing to control growth.

In some embodiments, depletion of proteolytic components is done by genetically engineering proteases in a host to degrade during the production of the lysate. For those proteases that affect growth and regulatory processes, host cells will be genetically modified to introduce tags that do not affect the protein but provide a residue that can be targeted during or after lysis creation. This can include non-destructive tags on the N terminus, C terminus, or the middle of the protein in between domains, including, but not limited to, polyhistidine (His6), maltose binding protein (MBP), calmodulin binding peptide (CBP), DYKDDDDK (SEQ ID NO.: 26) peptide (FLAG), glutathione S-transferase (GST), hemagglutin (HA), histidine-biotin-histidine (HBH), polypeptide tag from the cMyc gene (Myc), S-tag derived from pancreatic ribonuclease A, small ubiquitin-related modifier (SUMO), tandem affinity purification (TAP), thioredoxin (TRX), and V5 from a small epitope found on the P and V proteins of paramyxovirus of SV5. With non-destructive tags, after production of the lysate according to methods in Sun et al. (2013) JovE the tagged proteases can be removed from the system by column filtration of the processed lysate, antibody pull-down of the endonuclease and exonuclease, or other methods to deprive the system of the tagged molecule. This can also include destructive tags on the 5′, 3′, or the middle of the protein in between domains, including, but not limited to, ssrA, cln8, cln2, hsl1, UmuD, MerB. With destructive tags, the proteases must be protected from degradation during the growth of the cell, either by spatial localization (e.g., periplasm vs cytoplasm) or select control of the degradation enzyme that recognizes the degradation tag. Then, during lysis either spatial or select control of the degrading enzyme is released, and the targeted proteases degrade with an incubation step. The protease in question can also be reengineered such that the function is preserved, but a specific site is introduced such that it can be degraded after extract production. As an illustrative example, a target enzyme can be engineered to include a degradation tag exogenous to the organism that does not inhibit function and is not recognized by the host organism. However, upon creation of the lysate an enzyme that recognizes the degradation tag can be added, thereby removing the endonuclease and exonuclease in question. Methods that are described in U.S. Pat. Nos. 8,916,358, 8,956,833 are incorporated herein by reference in its entirety.

Protection of Peptides of Less than 110 Amino Acids in Length by Modifying the Peptide to Resist Proteolysis

While cell-free systems with minimal proteolytic ability can be used to protect peptides of less than 110 amino acids, there are many cases where the protection may not be sufficient. This includes (1) if the cell-free system is not as productive for catalyzing the reaction vs. a standard E. coli cell-free system (e.g., PURExpress yields are 7.5-20× lower than E. coli cell-free systems); (2) if the ribosomal natural product reaction cannot be catalyzed in the protease-limited cell-free system, due to lack of necessary co-factors, chaperones, or other additives to catalyze the reaction; (3) if the proteolytic ability of the cell-free reaction is still present; among others. One can also maintain the propeptide by modifying the peptide to resist proteolysis. In examples, we demonstrate the tagging of peptides less than 110 amino acids in length to prevent degradation in cell-free systems.

In some embodiments, the expressed protein of choice is fused to a partner protein tag, or a “stabilizing domain,” to prevent degradation, promote solubility, and aid purification. Many biologically important peptides are intrinsically disordered proteins and are thus vulnerable to proteolysis/degradation. These disordered proteins can be fused to stabilizing domains that are highly structured and soluble proteins to aid in the solubility and protection of the fusion partner. Further, some tags provide a “handle” for single-step purification. Examples of stabilizing domains that can be added to the 5′ or 3′ site, without limitation, include polyhistidine (His6), maltose binding protein (MBP), calmodulin binding peptide (CBP), DYKDDDDK (SEQ ID NO.: 26) peptide (FLAG), glutathione S-transferase (GST), hemagglutin (HA), histidine-biotin-histidine (HBH), polypeptide tag from the cMyc gene (Myc), S-tag derived from pancreatic ribonuclease A, small ubiquitin-related modifier (SUMO), tandem affinity purification (TAP), thioredoxin (TRX), and V5 from a small epitope found on the P and V proteins of paramyxovirus of SV5, N-utilizing substance A (NusA), green fluorescent protein (GFP), and ubiquitin. Novel protein tags can be chosen from extremely structured and stable proteins, or protein domains, using structure prediction analysis programs, such as PONDR (Predictor Of Natural Disordered Regions). The addition of a tag may affect the activity of the expressed protein or the ability for downstream enzymes to use the expressed peptide as a substrate. Therefore, the tag must be experimentally tested to confirm downstream utility or activity, and/or modeled to determine if downstream utility or activity is affected.

In some embodiments, the tag may be fused to the partner protein with a linker. The linker can be selected from one or more of a cleavable linker, a non-cleavable linker, a peptide linker, a flexible linker, a rigid linker, a helical linker, or a non-helical linker. In certain embodiments, the linker is a neutral linker that allows the stabilizing domain to be less likely to inhibit downstream utility or activity. The linker can be human serum albumin or an Fc domain, or a sequence comprising glycine and serine. Exemplary linkers can include, without limitation, Gly-Gly-Gly-Gly-Ser-Ser (SEQ ID NO.: 22), Gly-Gly-Ser-Gly (SEQ ID NO.: 23), Gly-Gly-Ser-Gly-Gly-Gly-Gly-Ser-Gly-Gly (SEQ ID NO.: 24), or any other combinations of Gly and Ser that is placed in between the fused domain and the protein or peptide coding sequence.

In some embodiments, the tag may include protease sites that allow the fused domain to be cleaved away from the protein or peptide. This may be necessary if the addition of the fused domain alters the conformation of the protein, interferes with the downstream applications of the proteins, or prevents the proteins from being crystallized, among others. Protease sites include, without limitation, Tobacco Etch Virus (TEV) sites, PreScission Protease sites, Thrombin Protease sites, Factor Xa protease sites, Enterokinase protease sites, among other sites. Those skilled in the art will be able to express a peptide using the tag, optionally purify out the peptide using an affinity tag or other method (e.g., size exclusion, FPLC), and then incubate the resulting solution with the protease tag followed by a size exclusion or other isolation method and an optional concentration method to have purified peptide without tag. This purified peptide can then be added in downstream cell-free reactions at high concentration.

In some embodiments, the tag may include other sites that allow the final fused protein to be detected by small molecule interactions, antibodies, affinity purification, or other reagents. These include FLASH/REASH sites, MBP, NusA, GST, His6, CBP, FLAG, HA, HBH, Myc, S-tag, SUMO, TAP, TRX, V5.

In some embodiments, the tag may be a combination of fusion proteins, fusion domains, natural linkers, proteases sites, and regions assisting detection by small-molecule reaction. This combination may occur on the 5′ end, 3′ end, or in the middle of the peptide.

Those skilled in the art will note that the tag serves to provide the input peptide less than 110 amino acids structure, thereby resisting proteolytic activity against the peptide. The addition of this tag may also assist the transcription and translation of the peptide, but any assistance is external to the main purpose of the tag, to resist proteolytic activity.

In some embodiments, the input peptide less than 110 amino acids can be modified to resist proteolysis, either through modification of side-chains on the amino acid, implementation of non-specific amino acids, or other modifications as described in Blaskovich, 2016 broadly for medicinal chemistry and specifically in Baumann et al., 2017 for natural products or D. Knappe, Henklein, Hoffmann, & Hilpert, 2010 for antimicrobial peptides, are incorporated herein by reference in its entirety. This modification can be installed synthetically, biochemically, enzymatically, or through a combination thereof. The resulting input peptide is resistant to proteolysis but can be modified by enzymes and other factors in the cell-free reaction composition, either endogenous or exogenously provided, to produce a final product.

### Example 1. Lasso Peptides Microcin J25, Klebsidin, Actinodyein, a Subclass of Ribosomal Natural Products, can be Produced in Cell-Free Systems

Microcin J25 is a model peptide in the lasso peptide family, first discovered in 1992 from a fecal-isolated E. coli strain. Like most lasso peptides, its synthesis involves 4 genes: mcjA (peptide precursor), mcjB (cysteine protease), mcjC (lactam synthase), and mcjD (ABC transporter) and only mcjA, mcjB, and mcjC are necessary for its biosynthesis in E. coli. Microcin J25's mechanism of action is two-fold, targeting the E. coli RNA polymerase and interfering with membrane stability. It has activity against E. coli (MIC 0.02 μg/mL), Shigella flexneri, and Salmonella enteriditis. The peptide demonstrated 3-fold decrease in Salmonella infection in mouse models, without inducing hemolytic activity.

Microcin J25 is representative of lasso peptide family (Hegemann, Zimmermann, Xie, & Marahiel, 2015). This family is relatively new, first discovered in 1991. Lassomycin analogs are phylogenetically distributed, ranging from gram-positive Streptomyces and Rhodococcus to gram-negative E. coli and thermophilic Thermobaculum. From 1992-2007, lasso peptides were mainly isolated from functional compound-driven screens (Hegemann et al., 2015) and this led to the identification of candidate therapeutics such as anantin (atrial naturetic factor antagonist), microcin J25 (gram-negative antibiotic), and siamycin (a HIV inhibitor). Since 2008, genome mining against a lasso peptide motif has led to the discovery of additional peptides yet to be characterized. Recent advances in the field include identifying critical regions of the peptide (Pan & Link, 2011), scaffolding peptide epitopes (T. A. Knappe et al., 2011), re-engineering the peptide for stronger antimicrobial activity (Pan, Cheung, & Link, 2010) and fusion-protein stability (Zong, Maksimov, & Link, 2015), and the identification of lassomycin (Gavrish et al., 2014). Since 2017, 1,300 (35×) more lasso peptides were identified from two new independent genome mining studies of which the vast majority have not been characterized. Total chemical synthesis has been unsuccessful at making functional protein (Lear et al., 2016) and heterologous expression is difficult. The actual number is likely larger than the reported 1,300: the Tietz et al. (2017) dataset primarily consists of lasso peptide clusters from Actinobacteria, while the Skinnider et al. (2016) dataset primarily consists of clusters from Proteobacteria.

The small 4.8 kb gene cluster size of microcin J25 is extremely conducive to testing in cell-free platforms. The sequences do not carry any risk factors for cell-free expression—none require known complex co-factors and none are membrane-bound. The sequence is well-defined and expressible in E. coli cells carrying mcjD. Combination of purified mcjA, mcjB, and mcjC produces functional microcin J25 in vitro (Duquesne et al., 2007). Expressing in E. coli cell-free avoids microcin J25's toxicity by inhibition of E. coli RNA polymerase (a T7 polymerase can be introduced to native E. coli RNA polymerase) and by membrane-based toxicity (cell-free does not require membranes).

Produced microcin J25 can be rapidly screened against known sensitive wildtype E. coli and non-pathogenic Pseudomonas spp., as well as known insensitive gram-positive Rhodococcus spp. and Streptomyces spp. 10 μg is sufficient for conducting 24 MIC assays at 200 μL scale. With cell-free expression yields of 0.75 mg/mL and ability to run 10 mL reactions, theoretical yields are ˜750× above required.

FIG. 6 shows an exemplary expression of the microcin J25 cluster in cell-free systems, the detection of the samples, and then a MIC to determine those with antibiotic efficacy. For those skilled in the art, it is apparent that if production of the microcin J2 cluster yields enough active microcin J25, that pooled sample detection is not necessary and samples can be detected directly. Those skilled in the art will also appreciate that many different methods can be used to assemble and express microcin J25, including assembly onto T7 promoters but also assembly onto sigma70 promoters or assembly using linear or plasmid regions.

We demonstrate that microcin J25 and produced is active in our cell-free systems. This is a surprising outcome, as lasso peptides, of which microcin J25 is an example, have not been able to be synthesized using synthetic chemistry techniques, as shown by reference to (Lear et al., 2016). SEQ ID NO.: 1, SEQ ID NO.: 3, and SEQ ID NO.: 4 show the promoter, utr, coding sequence, and terminator sequences of the mcjA (726), mcjB (727), and mcJC (728) expressed under sigma70 promoter, respectively in a E. coli cell-free system. It is understood that these can be tested as linear DNA as written, or can be tested as plasmids when cloned on a backbone (e.g., colE1, ampR). A control provided is SEQ ID NO.: 5, caulA (729).

When expressing combinations of 726, 727, 728, 729, and the plasmid pBEST-OR2-OR1-Pr-UTR1-deGFP-T500 (40019, Addgene), it can be seen in FIG. 7A that the combination of 726, 727, 728, and plasmid 40019 generates a knockdown of plasmid 40019 when read for deGFP channel fluorescence (485/515) after 12 hours of expression. All reactions contain 40019 in plasmid form at 2.66 nM, and the reaction is done in E. coli extract “eZS6” at 25% lysate, 75% buffer concentration, prepared as listed in (Sun et al., 2013) or “eAC15” at 30% lysate, 70% buffer concentration. GFP concentration was kept at 2 nM, and the other components kept at a total of 6 nM to control for competition affects. Hence, the condition for knockdown is 726, 727 and 728 in plasmid form at 2 nM, and the control expression (1) is no addition, (2) addition of 727 and 728 in plasmid form at 3 nM, and (3) addition of 729, 727 and 728 in plasmid form at 2 nM.

It is seen in FIG. 7B that adding purified microcin J25 to cellular reactions causes cell death with a MIC of 0.1 uM. If microcin J25 produced in cell-free systems is at high enough concentration, those skilled in the art will recognize that the MIC curve for killing live E. coli will be the similar. However, avoiding phage in the reaction is essential to ensure that the MIC is accurate and not caused by propogation of infecting phage carried over in a typical cell-free reaction that is contaminated with phage.

Another way to visualize the killing effect of microcin J25 produced in cell-free systems is through a high-throughput experiment shown in FIG. 8. In the top experimental setup, linear DNA templates are cloned that correspond to sigma70-UTR1-mcjA (726), sigma70-UTR1-mcjB (727), sigma70-UTR1-mcjC (728), sigma70-UTR1-caulA (729), and sigma70-UTR1-GFP (40019) using Golden Gate Assembly and additional methods as described in Sun et al. (2013) ACS Synthetic Biology. These are run in a E. coli-based cell-free lysate system in different concentrations, as listed by nM concentrations described in the chart, with all reactions containing 4 nM sigma70-UTR1-GFP and 3.5 gamS to protect linear templates. If microcin J25 is produced and folded properly, there should be a decrease in the activity of sigma70-UTR1-GFP as functional microcin J25 knocks down the activity of sigma70 RNAP. Through conducting a table of experiments, we are able to see that the expression of A, B, and C in one pot produces a statistically significant knockdown of GFP expression versus no DNA, only B and C (no propetide), or a propeptide that cannot fold (A′, or caulA).

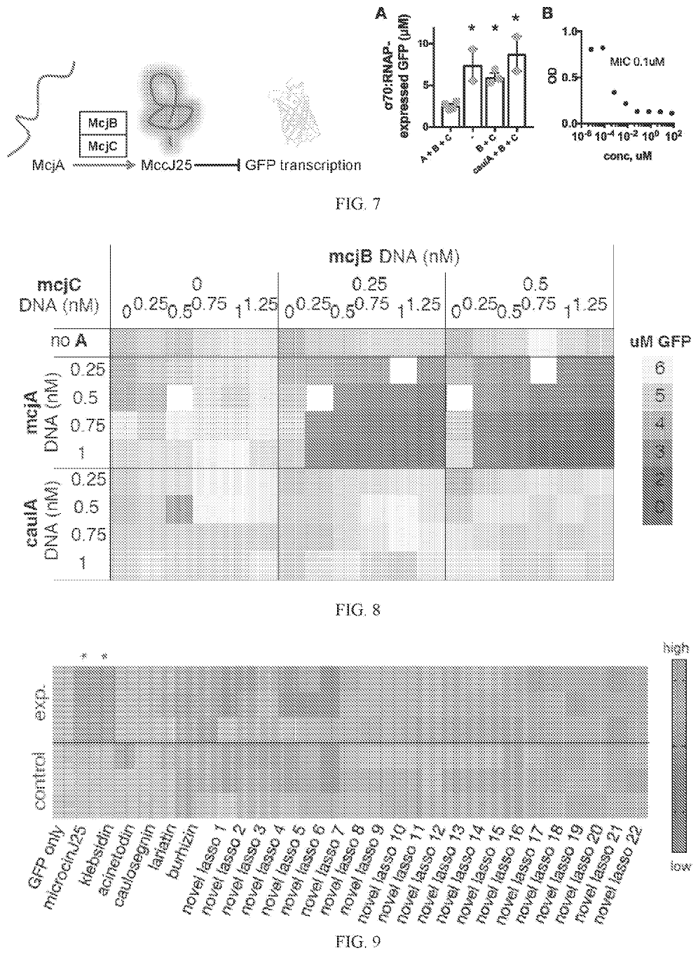

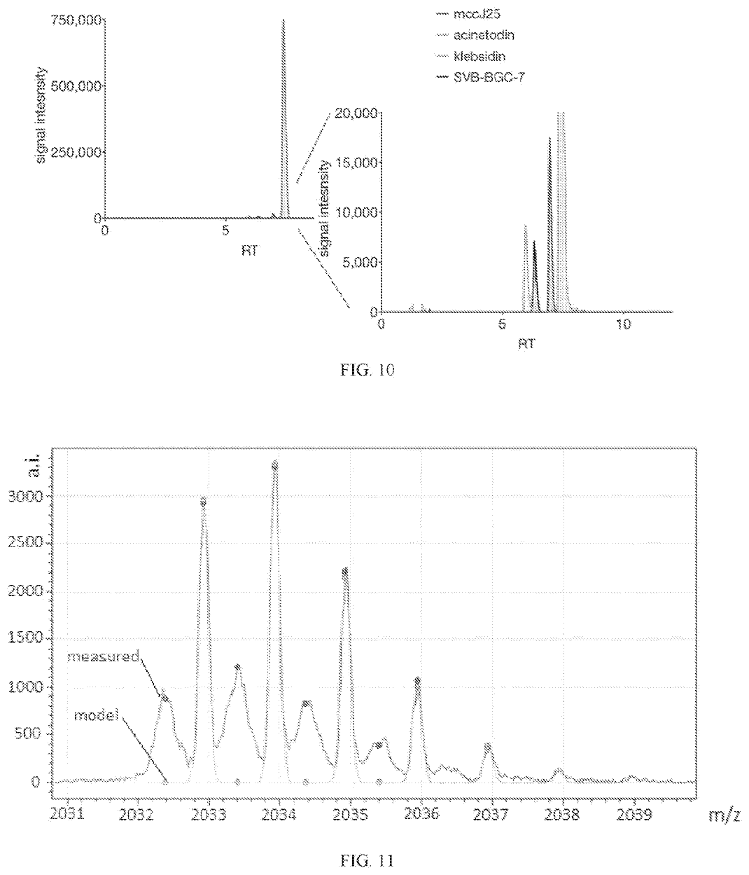

We were able to expand our functional assay as a screen for lasso peptides that are able to inhibit native RNA polymerase. We incorporated a panel of known lassos including those capable of inhibiting RNA polymerase and not, as well as a few predicted lasso clusters, as shown in FIG. 9. We were able to detect functional knockdown of native RNA polymerase by klebsidin and microcinJ25.

In the first round, the known lasso peptides microcinJ25, capistruin, burhizin and caulosegnin we obtained plasmids with the native coding sequences and cloned them into our expression system as parts. The number of individual coding sequences for each lasso cluster vary, but generally have a core of A, B (or B1) C and E (or B2). E is often fused to another gene. Our expression system incorporates these coding sequences individually with a sigma70 promoter, UTR and terminator. For the remaining known lassos that we screened; klebsidin, lariatin, and acinetodin, as well as a few predicted lassos, we synthesized DNA coding sequences to recapitulate the native peptides and assembled these synthetic coding sequences into our system. A GFP construct (40019 in linear) was assembled in the same way. Linear DNAs were generated either by PCR amplification from plasmid or the DNA assemblies themselves. We used 3.5 μM GamS to prevent template degradation in E. coli lysate. Reactions were run at 10 μl scale in a 384-well format. The reaction is done in E. coli extract “eZS6” at 25% lysate, 75% buffer concentration, prepared as listed in (Sun et al., 2013). Timecourses of GFP intensity were taken, and 12 hr endpoints were used to generate the heatmap. GFP intensity of lasso-cluster-containing reaction was normalized to their paired negative controls.

We expressed our GFP construct in the same reactions as our lasso constructs. GFP (4009 in linear) was expressed at 4 nM and the lasso cluster genes at 0.6 nM. For the negative controls we substituted the lasso A genes that code for the propetide substrates used to make functional lassos with an A gene from the caulosegnin cluster, which has been shown not to have RNA polymerase inhibitory activity. By comparing the GFP intensity of negative controls to reactions expressing the complete cluster, we were able to screen for RNA polymerase inhibition and our screen indicated that our klebsidin constructs assembled functional lasso. The specific sequences of the klebsidin constructs are 938 sigma70-klebsidinB (SEQ ID NO.: 6), 939 sigma70-klebsidinC (SEQ ID NO.: 7), 940 sigma70-klebsidinA (SEQ ID NO.: 8), and of the actinodein constructs are 908 sigma70-acinetodinC (SEQ ID NO.: 9), 909 acinetodinB (SEQ ID NO.: 10), 910 acinetoinA (SEQ ID NO.: 11).

From our functional screens, we demonstrate that microcin J25 and klebsidin are produced and active in our cell-free systems. Again, this is a surprising result given that these are characteristic lasso peptides that cannot be synthesized using synthetic chemistry techniques. We go on to show one can also detect lasso peptide production in cell-free systems even if there is no functional screen.

Shown in FIG. 10 is a sample detection of microcin J25, acineotin, and a predicted lasso, coming from a cell-free reaction. The microcin J25 reaction was conducted using sigma70-UTR1-mcjA (726), sigma70-UTR1-mcjB (727), and sigma70-UTR1-mcjC (728)) at 4 nM concentration of each; the acineodin reaction was conducted using 908, 909, and 910 at 4 nM concentration of each; the klebsidin reaction was conducted using 938, 939, and 940 at 4 nM concentration of each; and the SVB-BGC-7 cluster reaction was conducted using 931, 932, 932 at 4 nM concentration of each in a cell-free system that ideally has low amounts of polyethylene glycol (0.1%-0.2% w/v) and crowding agent (4% Ficoll 400). Reactions were run at 500 μl scale and peptides were separated by a liquid phase separation with butanol, dried, and resuspended at 25 μl. 5-20 μl of this sample was injected onto a Zorbax 300SB 4.6×150 mm column and eluted with a 74-2% water/acetonitrile gradient at 0.5 ml/min. Detection was accomplished by electrospray ionization into a quadrupole-time-of-flight mass spectrometer (qTOF). This demonstration confirms the production of microcin J25, acetineodin, and klebsidin, as well as a predicted lasso in our cell-free system, demonstrating that it does not need a functional assay to find new products and the results are generalizable.

Shown in FIG. 11 is a sample detection of klebsidin from a MALDI reaction. in a cell-free system that ideally has low amounts of polyethylene glycol (0.1%-0.2% w/v) and crowding agent (4% Ficoll 400). Reactions were set up as above, but at only 100 μl scale. The sample preparation following this was identical up to resuspension, which was to 5 μl total. Samples were incorporated into a 2,5-Dihydroxybenzoic acid matrix dried onto a ground steel plate. Matrix assisted laser desorption (MALDI) is able to detect klebsidin). This demonstration confirms that klebsidin is made in our cell-free system and that one can detect using multiple detection modalities.

### Example 2. Lactazole, an Exemplary Class from the Thiopeptide Class of Ribosomal Natural Products, can be Produced in Cell-Free Systems

Lactazoles are a novel family of thiopeptides, which are representative ribosomal natural products, isolated in 2014 from the genome mining of Streptomyces lactacystinaeus OM-6519. The resulting thiopeptides produced are macrocylic rings of 11 amino acids, with up to 56% post-translationally modified serine/threonine/cysteine residues. The lactazole biosynthetic gene cluster is a demonstration of the cell-free platform disclosed herein as a formerly cryptic cluster with minimal published data. The gene cluster is also short, spanning 9.8 Kb in size and composed of six genes.

Each coding sequence (lazA, lazB, lazC, lazD, lazE, lazE) has been synthesized and assembled onto sigma70 constitutive promoters using the methods outlined in (Sun et al., 2014). Set concentrations of these coding sequences, and tagged and untagged variants, and additives such as DMSO will be varied, each in a range of 1 nM to 16 nM, and expressed in cell-free systems as a reaction in a size range of 10 uL to 1 mL. The reaction will have low amounts of amounts of polyethylene glycol (0.1%-0.2% w/v) and will have another crowding agent (4% Ficoll 400). We will detect expression using both a qTOF LC-MS as well as MALDI and search for the three possible lactazole analogs using ion extraction of m/z 1401.3975 [M+H]⁺ for lactazole A, m/z 1,529.4586 [M+H]⁺ for lactazole B and m/z 1,176.2830 [M+H]⁺ for lactazole C³⁰.

Those skilled in the art will recognize that each coding sequence will need to be properly expressed in a cell-free system for the reaction to take place. To get each coding sequence to express, tags or additives may need to be added to the systems to ensure proper transcription and translation. In one example, we have expressed different variants of lazA, the propetide, under either a untagged version but with a T7 promoter replacing a sigma70 promoter (890/992, SEQ ID NO.: 12) or a tagged version with 5′ CAT (1071, SEQ ID NO.: 13). The results of expressing these constructs in PURExpress™, a system by NEB, that does not have proteolysis, are presented in FIG. 12. It can be seen that the untagged version (992) does not express compared to the tagged version (1071) in this system, indicating that there are secondary structure issues that impede transcription of the construct (rather than proteolysis issues). This indicates that in the final test, one will test not only the untagged version, but also different tagged versions of the construct and/or provide additives (DMSO) that can open up transcription ability.

LazC (773, SEQ ID NO.: 14) is experimentally found to express best at with 4% DMSO addition and no tag. LazD is experimentally found to express best as cat-lazD (897, SEQ ID NO.: 15), after testing multiple sequence variants of lazD (no tag 891, BCD2 tag from (Mutalik et al., 2013) 896, CAT tag 897, CAT tag plus ‘GGSG’ (SEQ ID NO.: 23) protein linker 898, CAT tag plus FLASH tag plus ‘GGSG’ (SEQ ID NO.: 23) protein linker 899, CAT tag plus his6 tag plus ‘GGSG’ (SEQ ID NO.: 23) protein linker 900, FLASH tag plus ‘GGSG’ (SEQ ID NO.: 23) protein linker 901) that can be assembled by those skilled in the art. This shows the need to test different variants of genes with different tags and different conditions in order to experimentally determine conditions that cause expression with little loss of activity. Expression of lazB (812, SEQ ID NO.: 16), of lazE (892, SEQ ID NO.: 17) and lazF (893, SEQ ID NO.: 18) is detectable without modification in an E. coli cell-free system.

Expression of the variants will result in E. coli cell-free reactions that will have detectable amounts of lactazole and/or lactazole intermediates. If detectable amounts are not made, but each gene is expressed and can be verified as active, the problem of expression may be due to the lack of cofactors in E. coli cell-free systems that are required for the production and activity of lactazole. If detectable amounts are not made, we will first utilize alternate cell-free systems, broadly made by gram-positive organisms, more specially actinomycetes, more specifically Streptomyces spp., more specifically Streptomyces lactacystinaeus OM-6519, in an attempt to supply the missing cofactors. This will involve utilizing cell-free systems that are non E. coli but are adept at transcription and translation, an example which is given later for Vibrio natrigens cell-free systems. If alternate cell-free systems fail, we will then utilize mixing 1%-50% of lysates of gram-positive organisms with our E. coli or other cell-free systems, more specially actinomycetes, more specifically Streptomyces spp., more specifically Streptomyces lactacystinaeus OM-6519, in an attempt to supply the missing cofactors. We will also purify specific cofactors that are known to affect lactazole production and add to the open cell-free systems, as isolated in (Hayashi et al., 2014), hereby incorporated herein by reference in its entirety.

### Example 3. Novel Ribosomal Natural Products, Especially Isolated from Human or Other Microbiomes, can be Produced in Cell-Free Systems

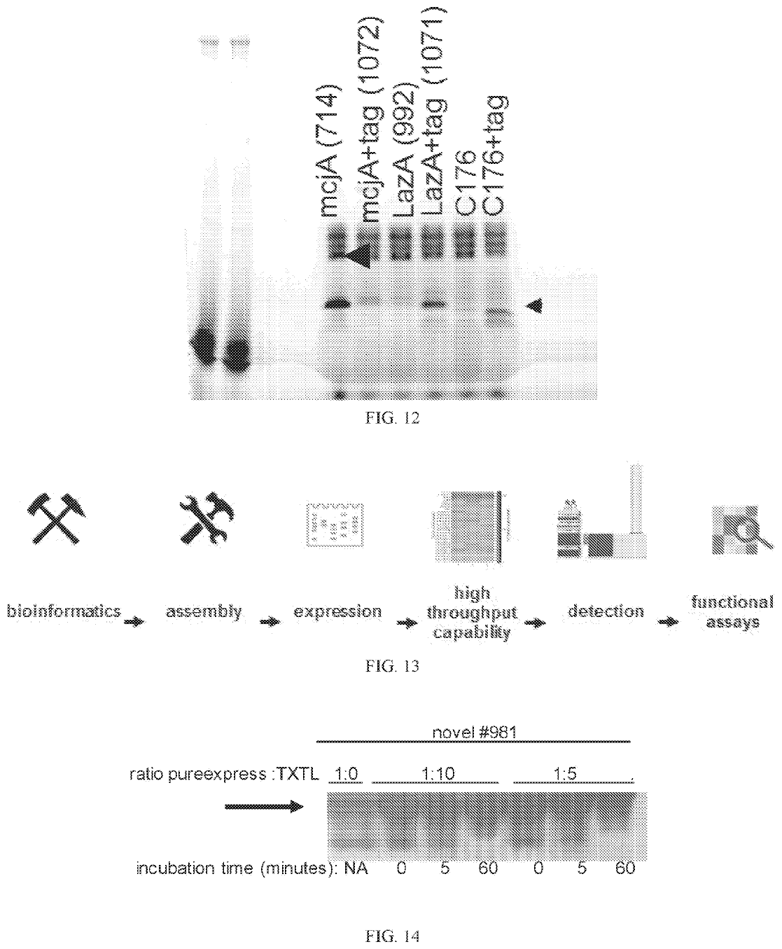

We demonstrate that novel ribosomal natural products can be produced in cell-free systems. The workflow for doing this is outlined in FIG. 13, where bioinformatically we can isolate novel natural products that we believe can be produced in cell-free systems, these products can be assembled in vitro, expressed off of linear DNA (or plasmid DNA) as individual constructs or as one operon, refactored (e.g., taking each coding sequence and putting on a constitutive promoter) or as one operon (e.g., taking a large operon and putting on one or multiple constitutive promoters). Expression can occur in E. coli cell-free systems, in non-E. coli cell-free systems, in PUREExpress™ or other purified protein systems, or in mixes of E. coli cell-free systems and lysates of other organisms or mixes of E. coli and non-E. coli cell-free systems. Expression can occur in low-throughput in 0.65 mL tubes to 50 mL conicals (e.g., 100 uL-10 mL reactions), or can occur in high throughput in 96-1536-well plate format (e.g., 1 uL-100 uL reactions), with care done to ensure oxygenation of reactions if oxygenation is required to drive ATP and cofactor regeneration. If there is a direct or indirect assay that can be directly read by a plate reader or other device amenable to high-throughput ability (e.g., by fluorescence, by fluorescence knockdown, Mg-aptamer signal, or secondary detection kits such as ATP assay kit (ab113849), NADP/NADPH Assay kit (ab65349), FAD Assay kit (ab204710), or similar), then the reactions can be done in high-throughput (e.g., assisted by traditional liquid handling, such as Biomek NxP, or assisted by acoustic liquid handling such as Labcyte Echo, or assisted by microfluidic or nanofluidic devises). If there is no direct and indirect assay that can be directly read, the successful expression can be isolated by analytical methods (e.g., LC-MS, GC-MS, MS/MS, qTOF, MALDI, HP-LC, SPR, NMR, UV, Western, chemiluninescence) or by running gels (e.g., SDS-PAGE). Functional assays can also be run (e.g., to check toxicity or activity) against known or unknown targets on the back-end, assuming the reactions have been optimized to produce enough product.

In a sample bioinformatics throughput, the current largest collection of automatically mined gene clusters is the “Atlas of Biosynthetic Gene Clusters”, a component of the “Integrated Microbial Genomes” Platform of the Joint Genome Institute (JGI IMG-ABC). IMG-ABC has annotations of 960,000 putative gene clusters from JGI's genome and metagenome datasets that are sorted by phylum and gene count. 217,395 clusters are from the phyla Actinobacteria. Of these, 33,364 clusters have the probability score of 1.0 and 18,202 are 1-20 genes in length. Only 311 of these were verified experimentally. Its gene cluster family network, comprising 11,422 gene clusters grouped into the main natural product gene cluster family of NRPS, type I and type II PKS, NISs, RiPPs, and TOMMs was validated in hundreds of strains by correlating confident mass spectrometric detection of known small molecules with the presence or absence of their established biosynthetic gene clusters.

For expressing natural products, we use lasso peptides as an exemplary example. From databases such as JGI-IGI, RODEO (Tietz et al., 2017), NCBI, PRISM, EMBL, ClusterFinder, antiSMASH, one can identify predictive lasso peptides by propetide sequence and other associated genes. We identified a set of 22 predictive lasso peptides using this approach, and using the workflow outlined for FIG. 9, expanded the set of predicted lasso clusters and refined our screen. We used lasso DNA concentration of 4 nM for the new screen (GFP was kept at 4 nM). We further observed that expression of the caulosegnin A construct compared to redundant controls where the A gene was omitted entirely were effectively identical and thus solely omitted lasso A genes in the negative controls. In a new batch of GamS 28 μM was determined to be the optimum working concentration. Reactions were setup at scale on a labcyte echo and GFP intensity was read with excitation at 485 nm and emission at 528 on a Biotek Synergy 2 plate reader. Otherwise experimental conditions were the same as before. We used our klebsidin construct as a positive control and recapitulated the result from the previous screen. We normalized the results here to negative controls and incorporated them into our heatmap as before. The results of this run are shown on the right side of FIG. 9. The heatmap demonstrates that the method can rapidly be used to screen for novel natural products as well as be used to show activity of controls. FIG. 10 demonstrates “Novel lasso 11” was found to have a m/z ratio at predicted peak, thereby indicating that the system can be used to screen for novel natural products.

### Example 4. The Propeptide is a Limiting Reagent in the Production of the Ribosomal Natural Product in Cell-Free Systems

FIG. 14 demonstrates the lack of degradation of a predicted ribosomal propeptide, ARVW01000001.1A_est (981, SEQ ID NO.: 2) in the PURExpress™ system that does not contain proteases. In this experiment, a PURExpress™ system is set up according to manufactuer's instructions with a saturating amount of T7-ARVW01000001.1A_est DNA (981). After 2 hours of expression, the product of the reaction is either not mixed at all (1:0, NA), or mixed with varying concentrations of E. coli cell-free systems (10%, 1:10, or 20%, 1:5) and incubated at 29 C for 0 min, 5 min, or 60 min. The products are then visualized on a SDS-PAGE using Comassie Blue staining, indicated by the black arrow. In the sample without proteases or when there is no incubation time, the propeptide can be clearly visualized. However, when E. coli cell-free systems are present, the product is rapidly degraded within minutes. Within the E. coli cell-free systems are proteases that are present and carried over from preparation of the cell-free system according to methods of Sun et al. (2013) JoVE. Therefore, without protective mechanisms, propeptides rapidly degrade in the cell-free systems.

### Example 5. The Propeptide can be Protected by the Utilization of TXTL Systems that are not Enriched in Proteolysis Components

We establish a method for determining what cell-free systems degrade propeptides by generalizing a screening approach, the results of which are shown in FIG. 15. A PURExpress™ system is set up according to manufacturer's instructions with a saturating amount of T7-ARVW01000001.1A_est DNA (981). 2.5 μL of the resulting PURExpress reaction was mixed with 2.5 μL cell-free protein synthesis reaction buffer, 0.5 μL candidate non-E. coli extracts, and water 2.83 μL water to roughly mimic cell-free protein synthesis reaction conditions. Samples were incubated in a 29° C. incubator for 1 hour. The 10% concentrations of lysates are from Pseudomonas fluorescens, Rhodococcus jostii, Streptomyces lividans, Streptomyces coelicolor, Vibrio natriegens, Tobacco BY2, Clostridium acetobutylicum, or HeLa whole cell extract. Unincubated control samples were prepared immediately before loading samples into a protein gel for visualization of peptide presence. The same proportions of the above incubated reactions were added directly to Bolt LDS Sample Buffer and Bolt Sample Reducing Agent to prevent any latent protease activity from degrading the peptide. To visualize the extent of degradation on a protein gel, 6.67 μL of the prepared samples containing PURExpress-expressed peptide and candidate non-E. coli extracts were added to 5 μL 4× Bolt LDS Sample Buffer, 2 μL 10× Bolt/NuPAGE Reducing Agent, and 6.33 μL deionized water and loaded onto a Bolt 4-12% Bis-Tris Plus gel. The gel was run at 200 V for 20 minutes and stained/destained with SimplyBlue SafeStain following manufacturer protocols, allowing visualization of the extent of peptide degradation. The products are then visualized indicated by the red arrow. Again, the propeptide can be clearly visualized in PURExpress reactions. However, for some lysates (Pseudomonas fluorescens, Streptomyces lividans, Streptomyces coelicolor, Tobacco BY2) the propeptide is degraded, whereas for other lysates (Rhodococcus jostii, Vibrio natriegens, Clostridium acetobutylicum, HeLa whole cell extract) the propeptide is partially still present. This demonstrates that different lysates have the ability to degrade propeptides that form ribosomal natural products, and this screen can be used to isolate lysates that do not degrade propeptides, that can then be produced into cell-free systems to catalyze the ribosomal natural product reaction.

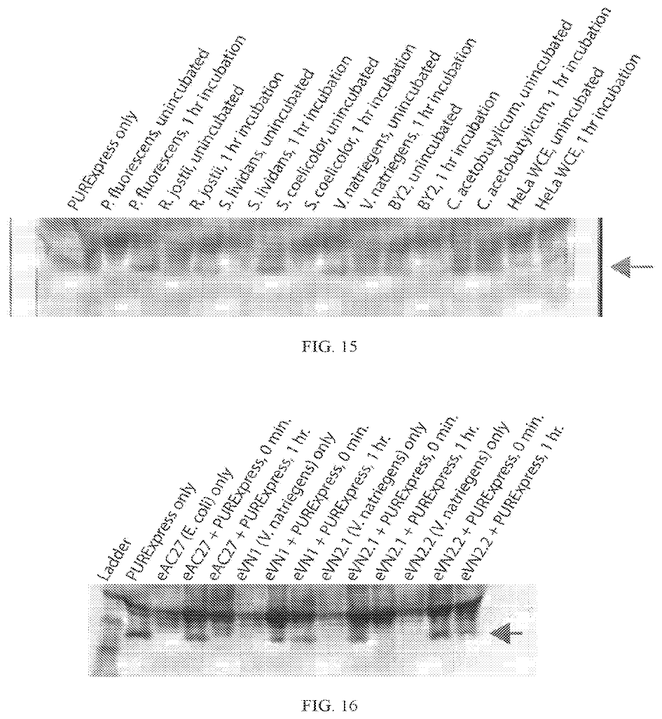

With the Vibrio natrigens cell-free systems produced, we test the systems using our PURExpress incubation assay. A PURExpress™ system is set up according to manufactuer's instructions with a saturating amount of T7-ARVW01000001.1A_est DNA (981). 2 μL of the PURExpress reaction is combined with 2 μL reaction buffer, 0.4 μL V. natriegens or E. coli extract, and 2.27 μL water to roughly simulate reaction proportions. The resulting mixture is incubated at 29° C. for 1 hour and loaded onto a protein gel to visualize degradation of the peptide band. E. coli extract in which the peptide is rapidly degraded or absence of extract served as controls. As seen in FIG. 16, all iterations of the V. natriegens extract demonstrate decreased proteolytic activity relative to eAC27, the E. coli extract. The aforementioned changes that improved protein yield in V. natriegens extract did not result in a corresponding increase in proteolytic activity.

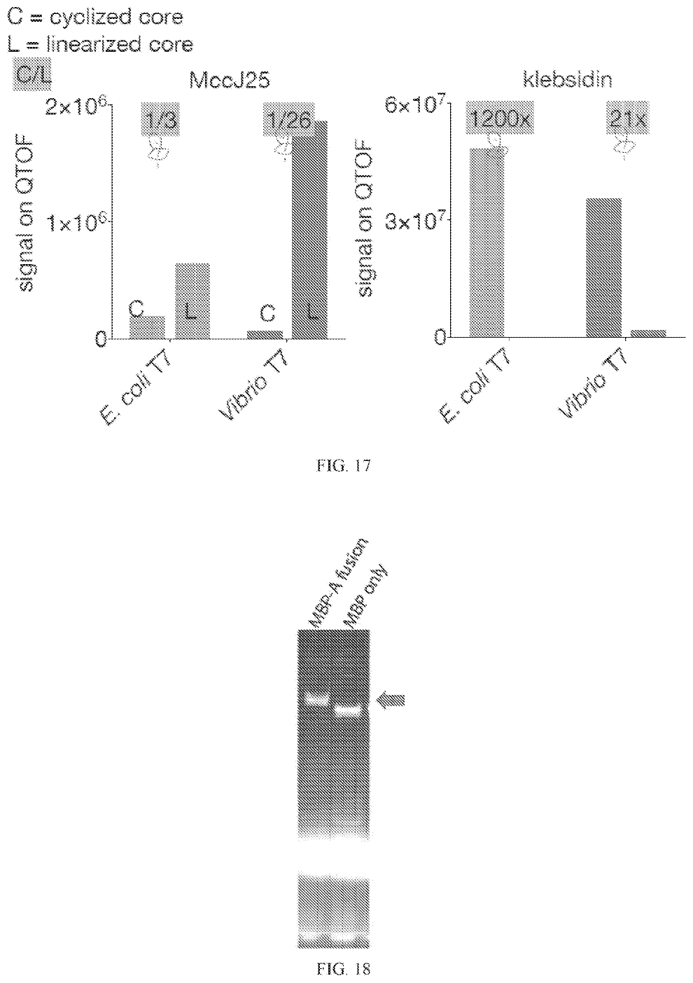

We demonstrate that by utilizing the produced Vibrio natrigens cell-free systems we are able to show increased processed pro-peptide present for both mccj25 and klebsidin in FIG. 17. Using previously described methods, we express mccj25 and klebsidin in either E. coli cell-free systems or Vibrio natrigens cell-free systems and using previously described LC/MS QTOF methods track the difference between linearized core, or the product of the “A” peptide (726, 940) being processed by the “B” enzyme (727, 938), vs. cyclized core, or the product of processing by “B” and “C” enzyme (728, 939). Linearized core m/z measured for [M+2H]⁺ at: MccJ25-L 1063.522, MccJ25-C 1054.517, klebsidin-L 1025.966, klebsidin-C 1017.465; all observations are within 10 ppm of calculated m/z values: MccJ25-L 1063.525, MccJ25-C 1054.519, klebsidin-L 1025.970, klebsidin-C 1017.466. Importantly, in the microcin J25 case Vibrio expression provides a large ratio of linearized core vs cyclized core (1/26 vs 1/3 ratio) compared to E. coli, indicating that significantly more linear peptide is available for processing. In the klebsidin case Vibrio cell-free expression leaves some linearized core present vs. E. coli cell-free expression cases that leave no linearized core present. This shows that the Vibrio natrigens cell-free systems are able to leave more linear peptides present. If a ribosomal natural product is tested in a product cell-free system that has little proteolytic degradation (e.g., Vibrio natrigens cell-free system), the final yields of the ribosomal natural product will be higher. In addition, we note that this experiment indicates that another limiting factor in producing natural products in cell-free systems is the native activity of enzymes, in the microcin J25 case “mcjC.” Conditions can be modified to improve enzymes for expression in cell-free systems, or enzymes can be evolved to improve activity.

### Example 6. The Propeptide can be Modified to Protect it from Degradation

We show that the propeptide can be tagged to prevent degradation in lysates or cell-free systems. In an example, mcjA (726) is known to degrade when expressed in E. coli cell-free systems. To stabilize mcjA, the peptide can be tagged either on the N terminus or the C terminus as a fusion protein that provides a stable domain that prevents proteolysis of mcjA. A linker and/or targeting region can be added to remove the tag. In an example, we tag mcjA with a maltose binding protein (MBP), generating a construct 1065 (SEQ ID NO.: 19) and compare the expression of 1065 to a wildtype MBP, 1066 (SEQ ID NO.: 20). In a SDS-PAGE gel expressing both constructs in FIG. 18, the expressed fusion protein was stable and easily visualized on a gel. The fusion protein was larger than MBP alone, indicating that the A propeptide was attached and not cleaved/degraded.

We would then test the ability of enzymes mcjB and mcjC to process the product of MBP-mcjA (1065), therefore producing the final microcin J25 lasso peptide by either detection on LC/MS QTOF or by activity assay. If mcjB and mcjC are not able to process MBP-mcjA, we would then switch the tag with other tag types (e.g., SUMO, GFP) and/or add neutral linkers to avoid interference with mcjB. We note that for the lasso peptide class, the “B” enzyme typically acts on the N terminus of the lasso peptide, thereby allowing tags to not remain on the final lasso peptide product. However, the propeptide can also be tagged on the C terminus, in which case the tag would remain on the final product (and may impede activity).

In another embodiment, the propeptide can be physically modified to prevent degradation. In particular, those in the art in lasso peptides recognize that while there are restrictions known on the donor residues and the acceptor residues for lasso peptides, other residues are open to modification. Therefore, on an open residue non-canonical amino acids can be implemented into the propeptide to protect it from degradation. For example, for capistruin, it is known that while T27 and G29 of the propeptide are critical for activity, as described (T. A. Knappe, Linne, Robbel, & Marahiel, 2009)11 and incorporated herein by reference in its entirety, other residues can be modified, e.g., with non-canonical amino acids, to prevent proteolysis. We would first determine if residues are critical or not critical for propeptide processing by downstream enzymes. Then, through synthetic peptide synthesis variants to reduce degradation can be generated and tested in the cell-free system for catalyzing downstream reactions.

### Example 7. Detection of Significant Ribosomal Natural Products from Cell-Free Reactions are Assisted by Cell-Free Reactions that have Low Amounts of Polyethylene Glycol (0.1% w/v) but Contain Crowding Agent

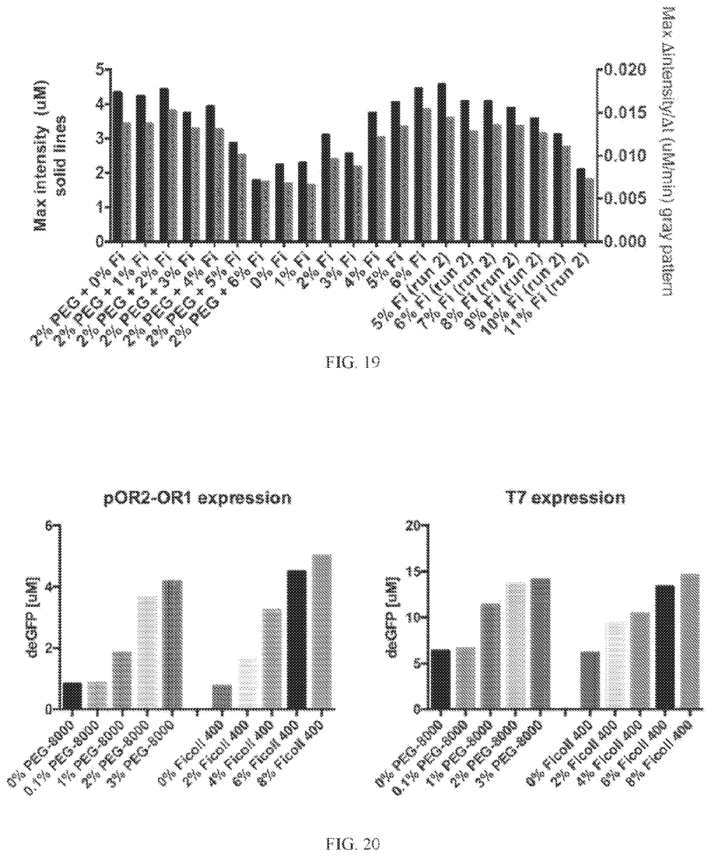

Crowding agents have been shown to be important in cells to assist protein-nucleic acid interactions and protein-protein interactions. To assist in protein-nucleic acid interactions and protein-protein interactions for catalyzing ribosomal natural products, one can supplement cell-free systems, that are not as crowded as cells, with crowding agents such as Ficoll, polyethylene glycol, polyethylene oxide, cyclodextrin, dextran, bovine serum antigen, glucose, among others. We show that in FIG. 19, supplementing a E. coli cell-free system with PEG or Ficoll 400 affects the final transcription and translation of GFP driven by a native promoter, with signal and maximum rate of expressing increasing with increasing concentrations of crowding agent addition. In addition, this benefit transcends different cell-free systems. In FIG. 20, varying concentrations of PEG-8000 and Ficoll 400 were tested in a V. natriegens extract prepared using previously described methods. The amount of GFP produced after 12 hours of reaction shows that V. natriegens extract is strongly dependent on added crowding agent for protein expression in both the case of native promoter expression off of the OR2-OR1-Pr promoter and off of T7 promoters (when T7 is supplemented into the reaction). While cases exist where crowding agent do not help expression, such as if reactions are not limited by crowding potential (e.g., very strong protein-nucleic interactions, very active enzymes), in many cases crowding agents will help catalyze ribosomal natural product reactions by assisting protein-nucleic acid and protein-protein interactions.

### REFERENCES

Arnison, P. G., Bibb, M. J., Bierbaum, G., Bowers, A. A., Bugni, T. S., Bulaj, G., et al. (2013). Ribosomally synthesized and post-translationally modified peptide natural products: overview and recommendations for a universal nomenclature. Natural Product Reports, 30(1), 108-160. doi.org/10.1039/C2NP20085F
Baumann, T., Nickling, J. H., Bartholomae, M., Buivydas, A., Kuipers, O. P., & Budisa, N. (2017). Prospects of In vivo Incorporation of Non-canonical Amino Acids for the Chemical Diversification of Antimicrobial Peptides. Frontiers in Microbiology, 8(32377), 503. doi.org/10.3389/fmicb.2017.00124
Blaskovich, M. A. T. (2016). Unusual Amino Acids in Medicinal Chemistry. Journal of Medicinal Chemistry, 59(24), 10807-10836. doi.org/10.1021/acs.jmedchem.6b00319
Buntru, M., Vogel, S., Spiegel, H., & Schillberg, S. (2014). Tobacco BY-2 cell-free lysate: an alternative and highly-productive plant-based in vitro translation system. BMC Biotechnology, 14(1), 37. doi.org/10.1186/1472-6750-14-37
Chiao, A. C., Murray, R. M., & Sun, Z. Z. (2016). Development of prokaryotic cell-free systems for synthetic biology. bioRxiv, 048710. doi.org/10.1101/048710
Duquesne, S., Destoumieux-Garzon, D., Zirah, S., Goulard, C., Peduzzi, J., & Rebuffat, S. (2007). Two Enzymes Catalyze the Maturation of a Lasso Peptide in Escherichia coli. Chemistry & Biology, 14(7), 793-803. doi.org/10.1016/j.chembiol.2007.06.004
Gavrish, E., Sit, C. S., Cao, S., Kandror, O., Spoering, A., Peoples, A., et al. (2014). Lassomycin, a Ribosomally Synthesized Cyclic Peptide, Kills Mycobacterium tuberculosis by Targeting the ATP-Dependent Protease ClpC1P1P2. Chemistry & Biology, 21(4), 509-518. doi.org/10.1016/j.chembiol.2014.01.014
Ge, X., Luo, D., & Xu, J. (2011). Cell-Free Protein Expression under Macromolecular Crowding Conditions. PLoS One, 6(12), e28707. doi.org/10.1371/journal.pone.0028707
Hayashi, S., Ozaki, T., Asamizu, S., Ikeda, H., Omura, S., Oku, N., et al. (2014). Genome Mining Reveals a Minimum Gene Set for the Biosynthesis of 32-Membered Macrocyclic Thiopeptides Lactazoles. Chemistry & Biology, 21(5), 679-688. doi.org/10.1016/j.chembiol.2014.03.008
Hegemann, J. D., Zimmermann, M., Xie, X., & Marahiel, M. A. (2015). Lasso Peptides: An Intriguing Class of Bacterial Natural Products. Accounts of Chemical . . . , 48(7), 1909-1919. doi.org/10.1021/acs.accounts.5b00156
Hong, S. H., Kwon, Y.-C., & Jewett, M. C. (2014). Non-standard amino acid incorporation into proteins using Escherichia coli cell-free protein synthesis. Frontiers in Chemistry, 2, 5949. doi.org/10.3389/fchem.2014.00034
Inokoshi, J., Matsuhama, M., Miyake, M., Ikeda, H., & Tomoda, H. (2012). Molecular cloning of the gene cluster for lariatin biosynthesis of Rhodococcus jostii K01-B0171. Applied Microbiology and Biotechnology, 95(2), 451-460. doi.org/10.1007/s00253-012-3973-8
Iwatsuki, M., Uchida, R., & Takakusagi, Y. (2007). Lariatins, novel anti-mycobacterial peptides with a lasso structure, produced by Rhodococcus jostii K01-B0171. Journal of Antibiotics, 60, 357-363.
Jewett, M. C., & Swartz, J. R. (2004). Mimicking the Escherichia coli cytoplasmic environment activates long-lived and efficient cell-free protein synthesis. Biotechnol Bioeng, 86(1), 19-26. doi.org/10.1002/bit.20026
Kelwick, R., Webb, A. J., MacDonald, J. T., & Freemont, P. S. (2016). Development of a Bacillus subtilis cell-free transcription-translation system for prototyping regulatory elements. Metab Eng, 38, 370-381. doi.org/10.1016/j.ymben.2016.09.008
Kim, D.-M., & Swartz, J. R. (2001). Regeneration of adenosine triphosphate from glycolytic intermediates for cell-free protein synthesis. Biotechnol Bioeng, 74(4), 309-316. doi.org/10.1002/bit.1121
Knappe, D., Henklein, P., Hoffmann, R., & Hilpert, K. (2010). Easy Strategy To Protect Antimicrobial Peptides from Fast Degradation in Serum. Antimicrobial Agents and Chemotherapy, 54(9), 4003-4005. doi.org/10.1128/AAC.00300-10
Knappe, T. A., Linne, U., Robbel, L., & Marahiel, M. A. (2009). Insights into the biosynthesis and stability of the lasso peptide capistruin. Chemistry & Biology, 16(12), 1290-1298. doi.org/10.1016/j.chembiol.2009.11.009
Knappe, T. A., Manzenrieder, F., Mas Moruno, C., Linne, U., Sasse, F., Kessler, H., et al. (2011). Introducing Lasso Peptides as Molecular Scaffolds for Drug Design: Engineering of an Integrin Antagonist. Angewandte Chemie International Edition, 50(37), 8714-8717. doi.org/10.1002/anie.201102190
Lear, S., Munshi, T., Hudson, A. S., Hatton, C., Clardy, J., Mosely, J. A., et al. (2016). Total chemical synthesis of lassomycin and lassomycin-amide. Organic & Biomolecular Chemistry, 14(19), 4534-4541. doi.org/10.1039/C6OB00631K
Modularity of RiPP Enzymes Enables Designed Synthesis of Decorated Peptides. (2015). Modularity of RiPP Enzymes Enables Designed Synthesis of Decorated Peptides. Chemistry & Biology, 22(7), 907-916. doi.org/10.1016/j.chembiol.2015.06.014
Mutalik, V. K., Guimaraes, J. C., Cambray, G., Lam, C., Christoffersen, M. J., Mai, Q.-A., et al. (2013). Precise and reliable gene expression via standard transcription and translation initiation elements. Nat Methods, 10(4), 354-360. doi.org/10.1038/nmeth.2404
Niederholtmeyer, H., Sun, Z., Hori, Y., & Yeung, E. (2015). Rapid cell-free forward engineering of novel genetic ring oscillators. eLife. doi.org/10.7554/eLife.09771.001
Ortega, M. A., & van der Donk, W. A. (2016). New Insights into the Biosynthetic Logic of Ribosomally Synthesized and Post-translationally Modified Peptide Natural Products. Cell Chemical Biology, 23(1), 31-44. doi.org/10.1016/j.chembiol.2015.11.012
Pan, S. J., & Link, A. J. (2011). Sequence Diversity in the Lasso Peptide Framework: Discovery of Functional Microcin J25 Variants with Multiple Amino Acid Substitutions. Journal of the American Chemical Society, 133(13), 5016-5023. doi.org/10.1021/ja1109634
Pan, S. J., Cheung, W. L., & Link, A. J. (2010). Engineered gene clusters for the production of the antimicrobial peptide microcin J25. Protein Expression and Purification, 71(2), 200-206. doi.org/10.1016/j.pep.2009.12.010
Shimizu, Y., Inoue, A., Tomari, Y., Suzuki, T., Yokogawa, T., Nishikawa, K., & Ueda, T. (2001). Cell-free translation reconstituted with purified components. Nature Biotechnology, 19(8), 751-755. doi.org/10.1038/90802
Shin, J., & Noireaux, V. (2012). An E. coli Cell-Free Expression Toolbox: Application to Synthetic Gene Circuits and Artificial Cells. ACS Synth Biol, 1(1), 29-41. doi.org/10.1021/sb200016s
Skinnider, M. A., & Johnston, C. W. (2016). Genomic charting of ribosomally synthesized natural product chemical space facilitates targeted mining. Presented at the Proceedings of the . . .
Sun, Z. Z., Hayes, C. A., Shin, J., Caschera, F., Murray, R. M., & Noireaux, V. (2013). Protocols for Implementing an Escherichia Coli Based TX-TL Cell-Free Expression System for Synthetic Biology. Journal of Visualized Experiments, e50762(79), e50762-e50762. doi.org/10.3791/50762
Sun, Z. Z., Kim, J., Singhal, V., & Murray, R. M. (2015). Protein degradation in a TX-TL cell-free expression system using ClpXP protease. bioRxiv, 019695. doi.org/10.1101/019695
Sun, Z. Z., Yeung, E., Hayes, C. A., Noireaux, V., & Murray, R. M. (2014). Linear DNA for Rapid Prototyping of Synthetic Biological Circuits in an Escherichia coliBased TX-TL Cell-Free System. ACS Synth Biol, 3(6), 387-397. doi.org/10.1021/sb400131a
Takahashi, M. K., Hayes, C. A., Chappell, J., Sun, Z. Z., Murray, R. M., Noireaux, V., & Lucks, J. B. (2015). Characterizing and prototyping genetic networks with cell-free transcription-translation reactions. Methods. doi.org/10.1016/j.ymeth.2015.05.020
Tan, C., Saurabh, S., Bruchez, M. P., Schwartz, R., & LeDuc, P. (2013). Molecular crowding shapes gene expression in synthetic cellular nanosystems. Nature Nanotechnology, 8(8), 602-608. doi.org/10.1038/nnano.2013.132
Thompson, J., Rae, S., & Cundliffe, E. (1984). Coupled transcription-translation in extracts of Streptomyces lividans. Molecular and General Genetics MGG, 195(1-2), 39-43. doi.org/10.1007/BF00332721
Tietz, J. I., Schwalen, C. J., Patel, P. S., Maxson, T., Blair, P. M., Tai, H.-C., et al. (2017). A new genome-mining tool redefines the lasso peptide biosynthetic landscape. Nat Chem Biol, 43, 9645. doi.org/10.1038/nchembio.2319
Zong, C., Maksimov, M. O., & Link, A. J. (2015). Construction of Lasso Peptide Fusion Proteins. ACS Chemical Biology, 11(1), 61-68. doi.org/10.1021/acschembio.5b00745

### EQUIVALENTS

The present disclosure provides among other things cell-free systems and use thereof. While specific embodiments of the subject disclosure have been discussed, the above specification is illustrative and not restrictive. Many variations of the disclosure will become apparent to those skilled in the art upon review of this specification. The full scope of the disclosure should be determined by reference to the claims, along with their full scope of equivalents, and the specification, along with such variations.

### INCORPORATION BY REFERENCE

All publications, patents and sequence database entries mentioned herein are hereby incorporated by reference in their entirety as if each individual publication or patent was specifically and individually indicated to be incorporated by reference.

1-25. (canceled)

26. A composition for in vitro transcription and translation, comprising:
a. a treated cell lysate derived from-E. coli;
b. a plurality of supplements for gene expression;
c. an energy recycling system for providing adenosine triphosphate and recycling adenosine diphosphate; and
d. an engineered propeptide operably linked to a stabilizing domain, wherein the engineered propeptide comprises one or more protease sites and the stabilizing domain prevents degradation and promotes stability of the engineered propeptide.

27. The composition of claim 26, wherein the cell lysate is depleted of proteases.

28. The composition of claim 26, wherein the plurality of supplements include reagents for transcription and translation.

29. The composition of claim 26, wherein the stabilizing domain is linked to the propeptide via a peptide linker selected from a group consisting of: (i) a peptide comprising Gly and Ser, (ii) Gly-Gly-Gly-Gly-Ser-Ser (SEQ ID NO.: 22), (iii) Gly-Gly-Ser-Gly (SEQ ID NO.: 23), (iv) and Gly-Gly-Ser-Gly-Gly-Gly-Gly-Ser-Gly-Gly (SEQ ID NO.: 24).

30. The composition of claim 26, wherein the engineered propeptide contains one or more protease sites that allow the stabilizing domain to be cleaved away, wherein the protease sites are selected from a group consisting of: Tobacco Etch Virus (TEV) sites, PreScission Protease sites, Thrombin Protease sites, Factor Xa protease sites, and Enterokinase protease sites.

31. The composition of claim 26, wherein the engineered propeptide contains a modification to resist proteolysis, wherein the modification is one or more non-canonical amino acids, a post-translation modification of an existing amino acid, or a stapled peptide.

32. The composition of claim 26, further comprising an engineered nucleic acid designed to express the engineered propeptide in the composition.

33. The composition of claim 26, further comprising an unstructured peptide provided at concentration of 0.1 mg/mL or higher.

34. The composition of claim 26, wherein the composition is designed to produce a natural product, a ribosomal natural product, a amatoxin, phallotoxin, bottromycin, cyanobactin, lanthipeptide, lasso peptide, linear azol(in)e-containing peptide, microcin, thiopeptide, autoinducing peptide, bacterial head-to-tail cycized peptide, conopeptide, cyclotide, glyocin, linearidin, microviridin, orbitide, proteusin, sactipeptide, toxin, or venom.

35. The composition of claim 34, further comprising one or more enzymes for modifying the natural product to produce a modified variant thereof.

36. The composition of claim 35, wherein at least a portion of the one or more enzymes are provided in the cell lysate.

37. The composition of claim 35, further comprising an engineered genetic circuit designed to express at least a portion of the one or more enzymes.

38. The composition of claim 34, wherein the natural product is further modified outside of the composition to produce a modified variant thereof.

39. The composition of claim 31, wherein the engineered nucleic acid is derived from a microbiome, human gut, animal, oral, skin, vaginal, soil, ocean, rhizosome, umbilical, vaginal, conjunctival, intestinal, stomach, nasal, gastrointestinal tract, or urogenital tract microbiomes.

40. The composition of claim 26, further comprising a crowding agent, present at no less than 0.1% (w/v), wherein the crowding agent is polyethylene glycol in the range of 0.1% (w/v)-0.2% (w/v).

41. A method of preparing a composition for in vitro transcription and translation, comprising:
a. providing a composition comprising:
i. a treated cell lysate derived from E. coli;
ii. a plurality of supplements for gene expression;
iii. an energy recycling system for providing adenosine triphosphate and recycling adenosine diphosphate; and
iv. an engineered propeptide operably linked to a stabilizing domain, wherein the engineered propeptide comprises one or more protease sites and the stabilizing domain prevents degradation and promotes stability of the engineered propeptoide;
b. determining that the composition is depleted of proteases;
c. providing an engineered nucleic acid to encode a propeptide; and
d. expressing the propeptide in the composition.

42. The method of claim 41, wherein the composition is depleted of proteases.

43. The method of claim 41, wherein the determining step comprises mixing the composition with an effective amount of a test peptide and determining that at least 10% of the test peptide remains after incubation for about 60 minutes.

44. The composition of claim 26, wherein the engineered propeptide contains a tag for detection by small molecule interactions, antibodies, affinity purification, or other reagents, wherein the tag is selected from a group consisting of FLASH/REASH sites, MBP, NusA, GST, His6, CBP, FLAG, HA, HBH, Myc, S-tag, SUMO, TAP, TRX, and V5.

45. The composition of claim 26, wherein the engineered propeptide is modified by the composition to produce a product, and wherein the engineered propeptide has a length of less than 110 amino acids.


SEQUENCE LISTING (26 sequences)

SEQ ID NO: 1 - DNA, 485 bp, unidentified
  misc_feature 1..485: note = Synthetic sequence
  source 1..485: mol_type = other DNA
aaaaccgaattttgctgggtgggctaacgatatccgcctgatgcgtgaacgtgacggacgtaacgaagacaaccacgcattgctgttctgagctaacaccgtgcgtgttgacaattttacctctggcggtgataatggttgcagcaagcaataattttgtttaactttaagaaggagatatacaatgattaagcattttcattttaataaactgtcttctggtaaaaaaaataatgttccatctcctgcaaagggggttatacaaataaaaaaatcagcatcgcaactcacaaaaggtggtgcaggacatgtgcctgagtattttgtggggattggtacacctatatctttctatggctgatgaactcaaagcccgccgaaaggcgggcttttctgtccttgagagtcgggcattgtcttcgctccttccggtgggcgcggggcatgactatcgtcgccgcacttatgactgtgttctttatcat

SEQ ID NO: 2 - DNA, 494 bp, unidentified
  misc_feature 1..494: note = Synthetic sequence
  source 1..494: mol_type = other DNA
aaaaccgaattttgctgggtgggctaacgatatccgcctgatgcgtgaacgtgacggacgtaacgaagacaaccacgcattgctgttcagatctcgatcccgcgaaattaatacgactcactatagggagacgacaacggtttccctctagaaagcaataattttgtttaactttaagaaggagatatacaatgaccaaacgcacttatgaaacacccgtactcgtcagcgcgggctcttttgcccgacgtacaggctcgggaagtccgaaggcggcccgtgatccgtttgggcgccgctggcttccttaatgaataactgaataggggatcccgactggcgagagccaggtaacgaatggatccccgagctcgagcaaagcccgccgaaaggcgggcttttctgtccttgagagtcgggcattgtcttcgctccttccggtgggcgcggggcatgactatcgtcgccgcacttatgactgtgttctttatcat

SEQ ID NO: 3 - DNA, 935 bp, unidentified
  misc_feature 1..935: note = Synthetic sequence
  source 1..935: mol_type = other DNA
aaaaccgaattttgctgggtgggctaacgatatccgcctgatgcgtgaacgtgacggacgtaacgaagacaaccacgcattgctgttctgagctaacaccgtgcgtgttgacaattttacctctggcggtgataatggttgcagcaagcaataattttgtttaactttaagaaggagatatacaatgatccgttactgcttaaccagttatagagaggatcttgttatcctggatataattaatgatagtttcagcatagtgcctgacgcaggtagcttgctaaaagaaagagataaattgcttaaagaattcccacaactatcttacttttttgacagtgaatatcatattggaagtgtttctcgtaatagtgacacttcttttcttgaagaacgctggtttctaccagaacctgacaaaacattatataagtgttctctatttaaacgatttatattattactcaaagtcttttactatagctggaatattgaaaaaaaagggatggcatggattttcataagtaataaaaaagagaataggctatactccttgaatgaagagcatcttatccggaaagaaattagtaatctttccattatctttcatcttaatatttttaaatctgactgtcttacctattcatacgcactaaaaagaattcttaattccagaaatattgatgctcatcttgttattggtgtaaggacacaacctttttatagccactcttgggtggaggttgggggacaagttatcaatgatgctcccaatatgcgggataaattatctgttattgcagagatatagtgaactcaaagcccgccgaaaggcgggcttttctgtccttgagagtcgggcattgtcttcgctccttccggtgggcgcggggcatgactatcgtcgccgcacttatgactgtgttctttatcat

SEQ ID NO: 4 - DNA, 1850 bp, unidentified
  misc_feature 1..1850: note = Synthetic sequence
  source 1..1850: mol_type = other DNA
aaaaccgaattttgctgggtgggctaacgatatccgcctgatgcgtgaacgtgacggacgtaacgaagacaaccacgcattgctgttctgagctaacaccgtgcgtgttgacaattttacctctggcggtgataatggttgcagcaagcaataattttgtttaactttaagaaggagatatacaatggaaatatttaatgtcaagttaaatgatacttcaattagaattattttctgtaaaacgctttctgccttccggacagaaaataccatcgttatgctcaaaggaaaagcagtttcaaatggcaaacctgtatccacagaggagattgccagagtagtggaagaaaaaggtgtttcagaagtaatagaaaatttagatggtgttttctgtatcctaatttatcattttaatgatctccttatagggaaaagcattcaatcaggccccgctctattttattgtaaaaagaatatggatatttttgtttcggataaaatttctgatatcaaatttttgaatccagatatgacattcagtctaaatataacaatggcagaacattatctgtcaggaaatcgaatagcaacccaggaatcactaatcactggcatttacaaagtaaataatggtgagtttataaaatttaataatcagttgaaacctgtgctacttcgtgatgagtttagtattaccaaaaagaacaattcaactatcgacagtatcattgataatattgagatgatgcgggataatagaaaaatagccctattattttccggaggattggattctgcattaatttttcacacacttaaagaatcaggtaacaaattctgcgcttatcattttttttctgatgaatctgatgacagtgaaaagtattttgctaaggaatactgttcaaaatatggagttgattttatatctgttaataaaaacatcaactttaatgaaaaactttatttcaatttaaatcctaatagtccggacgaaatccctttgatatttgaacagacagatgaagaaggtgaaggtcagccccccatagacgatgatttattatatctatgtggtcacggtggagatcatattttcggacaaaatccttcagaactttttggcattgatgcatatcgaagtcatggcttgatgtttatgcataaaaaaatagtagaattttccaatctcaagggaaagagatataaagatatcatattttcaaatatttccgcattcattaatacatccaacggatgttctccagcaaagcaagagcacgtatcagatatgaaacttgcctctgctcagttttttgcaactgattatacaggaaaaattaataaactaactccattcctgcataaaaatattatccagcattatgctggcttaccagtttttagtctatttaaccagcactttgatcgttatcccgttcgttatgaagcgtttcaacgatttggttcagatattttctggaaaaaaaccaaacggtcatcttcacagctaatattcagaattctatccggtaaaaaggatgaactagtgaatacaataaaacagtcaggattaattgaaatattaggcattaaccatattgaattggaaagcattttgtatgaaaatacgactacacgtctgacaatggaactaccatatatacttaacttataccgtctggcaaaattcattcaacttcaatccattgattataaaggttaatgaactcaaagcccgccgaaaggcgggcttttctgtccttgagagtcgggcattgtcttcgctccttccggtgggcgcggggcatgactatcgtcgccgcacttatgactgtgttctttatcat

SEQ ID NO: 5 - DNA, 437 bp, unidentified
  misc_feature 1..437: note = Synthetic sequence
  source 1..437: mol_type = other DNA
aaaaccgaattttgctgggtgggctaacgatatccgcctgatgcgtgaacgtgacggacgtaacgaagacaaccacgcattgctgttctgagctaacaccgtgcgtgttgacaattttacctctggcggtgataatggttgcagcaagcaataattttgtttaactttaagaaggagatatacaatgaccaagaagaacgccacccaagccccgcgtctcgtgcgcgtcggcgacgcccatcgcctgacgcaaggcgcgttcgtcggccagcccgaagcggtcaatccgctcggccgcgaaatccaaggctgatgaactcaaagcccgccgaaaggcgggcttttctgtccttgagagtcgggcattgtcttcgctccttccggtgggcgcggggcatgactatcgtcgccgcacttatgactgtgttctttatcat

SEQ ID NO: 6 - DNA, 985 bp, unidentified
  misc_feature 1..985: note = Synthetic sequence
  source 1..985: mol_type = other DNA
aaaaccgaattttgctgggtgggctaacgatatccgcctgatgcgtgaacgtgacggacgtaacgaagacaaccacgcattgctgttctgagctaacaccgtgcgtgttgacaattttacctctggcggtgataatggttgcagcaagcaataattttgtttaactttaagaaggagatatacaatgttcaacatgacacactacctgcgtttctccatttacaagaacgacctggtgatcattgatataaataacgacgaatatttcataatgaacgatgtgaatcacgagaatattagcttgctgactgatgtggaggaggaacttttagccgccggacttattcagtcactcaccccaattggaggcgataatgagaatttttacgatgaacgctggctcccgaggaaggcaatcttacgcaagattaatcctgtgcttttactgatggtttatagcatctttgtaaagtgtaaaaaaaacctggattctaatggctattatggtgtcatttacagtctgcagaacgttaaaaaaaaccaccgatgggataaatattcgcccggtgacattatcaattgcttaaactttattatgccgtttaaacattgcgaaaatccttgcctaatctattcatatgcactggttaccatgctgaaaaaagctacggggaaaggtacgctggtggttggtgttcgcactcgtccattcatcagtcatgcgtgggtagaactcgacggggaaatcatctccgataacatttatttgcgtgacaaactgtcggtaatcatggaagtgtgatgaataactgaataggggatcccgactggcgagagccaggtaacgaatggatccccgagctcgagcaaagcccgccgaaaggcgggcttttctgtccttgagagtcgggcattgtcttcgctccttccggtgggcgcggggcatgactatcgtcgccgcacttatgactgtgttctttatcat

SEQ ID NO: 7 - DNA, 1957 bp, unidentified
  misc_feature 1..1957: note = Synthetic sequence
  source 1..1957: mol_type = other DNA
aaaaccgaattttgctgggtgggctaacgatatccgcctgatgcgtgaacgtgacggacgtaacgaagacaaccacgcattgctgttctgagctaacaccgtgcgtgttgacaattttacctctggcggtgataatggttgcagcaagcaataattttgtttaactttaagaaggagatatacaatgttaattatcactggcaataaaaaaagcacgggcgcggataactatatttgcaagcataatatgtacatttatgcggatgagaactatgacacctgggattacaaagatacatacataatctttaaaggctattgttttgatgaggacggtaacggcattgccatcaataaagatagttttctcccggaagtcctggatcgcttgcccgaatttagtggatttttcgtgctcatcacaatctccaaagacaaaaccgttatatacaagagcttaagtcgcaacactgatgtcttttacagcgttgatgacaataacttgaccatctcggataatatcaaactgctgagtgaactgctcggcaaaaaaactattgatcctaaattttttagtagctttgttaataatgcatgggtcgcggtgttcctgtcgccgatttctggaattgaaaaaattaatggtggttgcaaatatgtttttgactctgctggggtgcacgtgtttaaacacgctaatttaacgccgaccaataaggacttcattgaagtgacctcaaatatgatcaaatcggtttgtaaaaataaaaaggtgtttttacatctgtctggcgggttcgactccacgtttattttttatatactaaaaaaaacgaacatcttcttcgaagtttatacccatgctcctgaccgttacgataatgattcagaagtgaatcgtgtccgggaactttgcactaagaataacgtaccgtttcgtgttgtgagtggttttccagatattctcaaatctaacaaagaagtgtcaataccctctgacgtcaatgtgattgagaacgaatccgaggacaaccagtacaacgagctgttaaacaaccacgatatcgtattcttgaacggccatggcggggactgtctatttgtgcaaaacccatcactgaagtccgtacaccatcgtctgaaacacggacgtttgattaagggtttgtgcaacgcttataaactgtgccgccttaagtatctttctttcacagagatcatcaatccaagaagccggattcattgcaacaactggtttagcgacacaaaatataaaggtttctaccagcatccgctgctgatcaacatcgatgattcgtcaccggaatatgaccatattgccaacatgctgtactttatggagtcactgcctctgcaactgaaggggggagcaatgatgttcagcccatttcttatgagctgtgcatttcgggtatttatgaaatataggtatgacgataatttctcatccgagcacgatcgcattctcgcccgaaaaatcgcctacaacattgcgcatgatatccaactgttcgatgtacgtaaacgctcgtccaacaatctgctgttcgactttctgcataagaataaggaaaagattcttttgctgatcaaccgaggcttcacacagggtatgggtgaggtaaccaccgatgatctgaaagaatcgttagaaattaataccagtattgggatagatggtaatgcgacgaaattcctgaaactgatgatgttaaaccgctatgcagaaatgaatatgcttacgaaagagtagtgaataactgaataggggatcccgactggcgagagccaggtaacgaatggatccccgagctcgagcaaagcccgccgaaaggcgggcttttctgtccttgagagtcgggcattgtcttcgctccttccggtgggcgcggggcatgactatcgtcgccgcacttatgactgtgttctttatcat

SEQ ID NO: 8 - DNA, 505 bp, unidentified
  misc_feature 1..505: note = Synthetic sequence
  source 1..505: mol_type = other DNA
aaaaccgaattttgctgggtgggctaacgatatccgcctgatgcgtgaacgtgacggacgtaacgaagacaaccacgcattgctgttctgagctaacaccgtgcgtgttgacaattttacctctggcggtgataatggttgcagcaagcaataattttgtttaactttaagaaggagatatacaatgatgcaacagaaaaaaaacgatatgaaaaaggtaacgcttaagaaactaaacaagagagcctcgaaagttactcggggtagtgatggccctatcattgaatttttcaaccccaatggggtgatgcactatggttagtgaataactgaataggggatcccgactggcgagagccaggtaacgaatggatccccgagctcgagcaaagcccgccgaaaggcgggcttttctgtccttgagagtcgggcattgtcttcgctccttccggtgggcgcggggcatgactatcgtcgccgcacttatgactgtgttctttatcat

SEQ ID NO: 9 - DNA, 2050 bp, unidentified
  misc_feature 1..2050: note = Synthetic sequence
  source 1..2050: mol_type = other DNA
aaaaccgaattttgctgggtgggctaacgatatccgcctgatgcgtgaacgtgacggacgtaacgaagacaaccacgcattgctgttctgagctaacaccgtgcgtgttgacaattttacctctggcggtgataatggttgcagcaagcaataattttgtttaactttaagaaggagatatacaatgatcttcctccgccgtttggtagaaaaagatgagcttagcctaaacaagaataattcttacaagtccaaagtgtttgataatttctacttcgaacatgataccagttttgatgtttgcgaacatgacaatttttgtgtgattttcaatggtgaggcgttttcctacaccagcgcaaaatcgctcaaaagtatcctgtttgaggtaggcagtgtcgagctgttgctgaaagaaatatctgatttagttggaaaatatactattctgatcatctacaaaaatacactgaacgtcattcagtctctgtataagaacatagatatctattatatggtggagaatgatgaattttacgttaccagtaatctgagcgcgttgctgtcgactgggaattttgaaattgacaccagttatgcgtacagctttattttgaacaagagaaactttggccttgaaaccccttttaaaaacgttaaaattgtgccccaaggtgagtcttgcttgtttaagtcctccaacaatataagccgaagcttggtttctttccgcgaaatttcaaaagtgaactacatcgaagagattagcaaaaacattaatctgtttaatctggataaagactcaccgctgtatgtcagctttagtggtggcttggactcttcggtagttttatattctgcactgatggccaactttcgtccaaaagccatacacctgctgccgcctgaggaacactccagcgaattagaaattgctcagtcagtggcaaaagaatttgacgtggaattacttgtattcaagcatccgtatttcttatcaggtcgggaaacattccagccgtccaccctgacatataatatttcagacatccacattctgtcaagtggactatgtgaaacttctgaaatttataatgatttaatgaaatcggaagccctgccgctgggccttacgctcacgggtcagggtggcgatttcattttcatgcaaaacccgcccgaaaatatcgggttcgatgcgctgaaagaaaaaggcttacggagcttcctgaacaagacaaaacagtatagcctgttgaaaaaggctcgctacttcaacatcctgcacaaaaatataaaaaactttatcggtaaccttttttcagacacatcggtttataacaacttctttgacaacaacgtgactttgagtgaatccaaacatctgctcttagactcagtttcgaataagtcggctaaatacgattatattcatggggtcctgacgggactttatagtgtaaaacctactcgcaacgatggatataaaatcctccacccgctgtatctgcaaaatattgtgtttaatatcattaacaatagcgtttgtgatatgtttaacgaggaccatgataggctgatcattcgagaacagttttatcgtaaaagtcagagcgaggtcgcgtggcgcaaaacgaagagatcttcctccggtgccctctttatgtttcttaataacaatcgcgaattaatttacgattatctgctcaacggctacgtagtggataccctgaaaatcaataagaaatggctggacgaggaaatttctcagaatacgatggttggcatcacgaacaactgcggcattgtgatgaatatcctgcaactggagatctactatcgccagttcgtgtggcgtgtcaaatgatgaataactgaataggggatcccgactggcgagagccaggtaacgaatggatccccgagctcgagcaaagcccgccgaaaggcgggcttttctgtccttgagagtcgggcattgtcttcgctccttccggtgggcgcggggcatgactatcgtcgccgcacttatgactgtgttctttatcat

SEQ ID NO: 10 - DNA, 1027 bp, unidentified
  misc_feature 1..1027: note = Synthetic sequence
  source 1..1027: mol_type = other DNA
aaaaccgaattttgctgggtgggctaacgatatccgcctgatgcgtgaacgtgacggacgtaacgaagacaaccacgcattgctgttctgagctaacaccgtgcgtgttgacaattttacctctggcggtgataatggttgcagcaagcaataattttgtttaactttaagaaggagatatacaatgccgtataaattccgaaacaacattaaagctacgatctgcgaggacgacctgatcattttggacatagagaaagatgcctattacttaattaataacattaatgcggatttaatcaacaactttctgaaaggggtagatcatcacgatctttcagacatcctcaactatgactggctgaacgaatccgaaaatatttacgatcagcttatagccaaaaaattaaaaggttactttgagcaacgttggattacacacaacatcttttgtgagaaattgaattttaagatatatctgaagataattattgaaatcttcaagattaactttaaaattcagaaactggattttaataccattagtaacctgaaaaataggggctctatcgaggattttagcggcaactacgaagatatcatttattcagttgttagtagcctcaacaaattatttgtgctggatatgtcgaataataaatgtctggcgtattctttcattcttttcaagatcctcaagagctacggaatcaatgcagtgctgaaaattggtgtgcgcactaagccatttttctcgcatgcgtgggttgaagtagataaaaaagtgctgagcgacgtccagatggtcaatgaaaatttggccatcattatggaattcagttaatgaataactgaataggggatcccgactggcgagagccaggtaacgaatggatccccgagctcgagcaaagcccgccgaaaggcgggcttttctgtccttgagagtcgggcattgtcttcgctccttccggtgggcgcggggcatgactatcgtcgccgcacttatgactgtgttctttatcat

SEQ ID NO: 11 - DNA, 520 bp, unidentified
  misc_feature 1..520: note = Synthetic sequence
  source 1..520: mol_type = other DNA
aaaaccgaattttgctgggtgggctaacgatatccgcctgatgcgtgaacgtgacggacgtaacgaagacaaccacgcattgctgttctgagctaacaccgtgcgtgttgacaattttacctctggcggtgataatggttgcagcaagcaataattttgtttaactttaagaaggagatatacaatgaaaaatttgaacaaaatgtttaagaaagaacaaaagtaccataaacagctaaaggtaatcagcgtgaaaggtagtgcctctaaaatgacactgggagggaaaggccccatatttgaaacctgggtgaccgagggcaactattacggttaatgaataactgaataggggatcccgactggcgagagccaggtaacgaatggatccccgagctcgagcaaagcccgccgaaaggcgggcttttctgtccttgagagtcgggcattgtcttcgctccttccggtgggcgcggggcatgactatcgtcgccgcacttatgactgtgttctttatcat

SEQ ID NO: 12 - DNA, 304 bp, unidentified
  misc_feature 1..304: note = Synthetic sequence
  source 1..304: mol_type = other DNA
aataattttgtttaactttaagaaggagatatacaatgtccgacatcacggcgagccgggtggaatccctcgacctgcaggacctggacctgtccgagctgaccgtgaccagcctgcgcgacacggtggcgctcccggaaaacggcgcgtcctgggggtcctgctcctgccaggccagcagcagctgcgcccagccgcaggacatgtaatgaataactgaataggggatcccgactggcgagagccaggtaacgaatggatccccgagctcgagcaaagcccgccgaaaggcgggcttttctgt

SEQ ID NO: 13 - DNA, 563 bp, unidentified
  misc_feature 1..563: note = Synthetic sequence
  source 1..563: mol_type = other DNA
aaaaccgaattttgctgggtgggctaacgatatccgcctgatgcgtgaacgtgacggacgtaacgaagacaaccacgcattgctgttcagatctcgatcccgcgaaattaatacgactcactatagggagacgacaacggtttccctctagaaagcaataattttgtttaactttaagaaggagatatacaatggagaaaaaaataatgtccgacatcacggcgagccgggtggaatccctcgacctgcaggacctggacctgtccgagctgaccgtgaccagcctgcgcgacacggtggcgctcccggaaaacggcgcgtcctgggggtcctgctcctgccaggccagcagcagctgcgcccagccgcaggacatgtaatgaataactgaataggggatcccgactggcgagagccaggtaacgaatggatccccgagctcgagcaaagcccgccgaaaggcgggcttttctgtccttgagagtcgggcattgtcttcgctccttccggtgggcgcggggcatgactatcgtcgccgcacttatgactgtgttctttatcat

SEQ ID NO: 14 - DNA, 1588 bp, unidentified
  misc_feature 1..1588: note = Synthetic sequence
  source 1..1588: mol_type = other DNA
aaaaccgaattttgctgggtgggctaacgatatccgcctgatgcgtgaacgtgacggacgtaacgaagacaaccacgcattgctgttctgagctaacaccgtgcgtgttgacaattttacctctggcggtgataatggttgcagcaagcaataattttgtttaactttaagaaggagatatacaatgtctgatcctgctgatgggcgtggggccgtaacggcgtgggacgtggtattgtatcactatcgcccggataaggctcgcgcattacgcgaggcagttcttccattggcccgtcaggctgctgccgaaggattggccgcacacgtggagcgccactggcgtttcggcccacatcttcgcctgcgcttgcgtggtcctgaggcgcgtgttgctggggccgcccagcgtgctgcagaggcgctgcgcgcatgggcagcggcacacccgtcagtagccgatcgctctgatgagcaattattagccgaagccgcagtagccggacgtgcagaactgattgccccaccctatgctccccttgttccagataacaccgttgttgctgcgccagcagaccgctcggcggaggacgcacttcgtgcgttaattggtgctgaatccgctgagttacgtgaggagttgcttcgtacgggtttaccggcattggactccgcttgccacttcctgggggcgcatggggatactccccaagcacgcgtacaattagtggtaacagcgcttgctgcccatgccacggcccaccccgacggactggttggagcccattactctgtgctgagtcatcttgaggatttcttagttcacgaagatcccgacgggagtctgcgtgcagccttcgaacgccgttgggaacagtcgggtcgcgccgtcacggcattggttggtcgtattgccgacgggggggcgcgtgattgggaacgtgattgggcacactggtcggcgactgcttggtctttggccgagcgtcgcttaacagcgggcgccgatctgggtggtcgtcacgcggagtaccgtgaacgcgcagaagcgcttggcgaccctgcaacagccgaacgttggaacgcggaactgcgcacccgttacagcgagtttcatcgtatgttacagcgtgcggaccctgatggacgcatgtggcaccgccccgactacttgatcaatcgtgcgggaaccaatggtttgtaccgcttgttagctatctgcgatgtacgccctatggaacgctatcttgcagcgcacttgctggtacgcagtgttccggagcttacagggcatcgttggcagactctgctgggggctgcagagcaaccgggcggccctgagcagagtggcgcggctggcgctacgggcggggctggccgtaccaaactggaaggtgctgcctgatgaataactgaataggggatcccgactggcgagagccaggtaacgaatggatccccgagctcgagcaaagcccgccgaaaggcgggcttttctgtccttgagagtcgggcattgtcttcgctccttccggtgggcgcggggcatgactatcgtcgccgcacttatgactgtgttctttatcat

SEQ ID NO: 15 - DNA, 2080 bp, unidentified
  misc_feature 1..2080: note = Synthetic sequence
  source 1..2080: mol_type = other DNA
aaaaccgaattttgctgggtgggctaacgatatccgcctgatgcgtgaacgtgacggacgtaacgaagacaaccacgcattgctgttctgagctaacaccgtgcgtgttgacaattttacctctggcggtgataatggttgcagcaagcaataattttgtttaactttaagaaggagatatacaatggagaaaaaaataatgaccgctgaaccggacgctgttcgcccgcgtctgcgccctggcgttgctgtcaccccactgcgtgaggggcttcatctgcgtggccgcgagagctcagtgacgcttgaagggagtcgcgcattgcctgcgctgtggcaagtattagccgcccgcttggggccgcaggccgaggcagctgacgcggccgttgaagctactgtggaacctcgtgtggcggctgctttagccactgtaacggcacgcttacgtgagcacggtttgctggtggatcatccggacggagtccgcctgcccccttggccaggagcggttgcggatgacccgggaggagctgaagcggcgttagcagcagcccgtcctgtggtggctgcggcagaccctgatggtccgtcggcacgtgctatggcacgcgcgttggcacgtggaggtactgccgcccccgcagttgtagcagagcctgggttaccagctgggcgtgttgtagcaactgccgatggccctgccggcactgaattagctgtagcagtccaatgcggggccgatggagggtttgtcactgaaccagcagatcccgcccgcgcccgtacagacgcggcggcgcttgcagcccgtttggaacccgcccctgtcgctgatccgccaccggtgcttctggcacttcttgctgctgcaggggctcagcgtctgctttgtgcagttgcaggtttacctgatccaggagagccggccgacgaccctcgccttctggacggccgtcctaccgttcttatcgcagacgctgcgcctccgcacgctgaacatcacccgtgggctgcaggtccgggtgctgttgcggcgcctcctggttcattggcagaggccctgcgccgtgtcaatgccttgggtgatccccgtcttggtgttttggacgccccctcagccggcgacttgcctcaattgcctgtcgctcttgtctcttgtgctacccctgcaggcccgctggcagcaggggctgtccgcactgatttggcacgccttgcggcagcctgccgtagcgcggaacttcatttggcagcggtggggggaggtgccgtgccggtagttggagttgacccagaccacgctttagggctggcacttcgtcgcgcagtacttgcacgcgctgtgcgtggggatcgtccattacccgacgatcgtgctgctcgtggggaccgcacagtgcctgaaagtgcctggcgtgaacatccacaggccggtcattggtatggtgtgttagcccgtcgcctggggcgtgccccagagcccacaattcgtcagctttcaggagaaagtgtgtatttggcccaggtagaggaaggccgcgctgtagaggctacgcccgcggacgcggttgctcacgcggccttggcagcgttaacacgcctgatggcacgtggggcggggttggcggccgtgcatcatacggtactttcgggtgccgcagctcctttagcggctgcgggtcgcacacctgcagcctggactgatctgggctgggcggatcgttggcttgctgatattgcagaccgcgaggccgacctgcatgcagcattagtccgtatcactggacttcgtacagcacgctggcgccccgctacgcctgaggctcgcccattcgcagatgcattggacggctgtgggtttaccgcgttaacagcagagggtgggcgcccatgatgaataactgaataggggatcccgactggcgagagccaggtaacgaatggatccccgagctcgagcaaagcccgccgaaaggcgggcttttctgtccttgagagtcgggcattgtcttcgctccttccggtgggcgcggggcatgactatcgtcgccgcacttatgactgtgttctttatcat

SEQ ID NO: 16 - DNA, 3010 bp, unidentified
  misc_feature 1..3010: note = Synthetic sequence
  source 1..3010: mol_type = other DNA
aaaaccgaattttgctgggtgggctaacgatatccgcctgatgcgtgaacgtgacggacgtaacgaagacaaccacgcattgctgttctgagctaacaccgtgcgtgttgacaattttacctctggcggtgataatggttgcagcaagcaataattttgtttaactttaagaaggagatatacaatgcccaaccgcgcagcacctccccgtgatgcacgcgcacctgtgccagcgcccgctccagcagcctacgcagcgcgtcacgctctggtacgtagtactgtattggcgtggccagcccaaagcgctgcaactgcacacacccgcgctctgctgcgtgatctggcggccgctgaggctgctgctgaagcactgcgtcccgccttgtgtgatgacttgtatgcgggacgtgctggtcacgatgaggaatttcatcgtcgtgttgtccttccgctgcgccgtgaccttcacaacggtcgtacacctcgtgcagcattattagatcgtttggcagacttgccacgtcgcattccgcgcttagcagagtggcttgagcttcgccgtttgcgcgctcgcttattagatgctctggctgatgctgttccacctgctttagcagcagaacgcgcggcattggcggatatttgccgtgagcccgtattcactcgtgctgtagcgctgaccagtgctgatttgttgcgcgctgtggcgaatactgcgggtgctactggtgagccgcctcgcggccgtgcgcgcaaggaggaggcagctgtcctgcgccacgcccttcgcgctacagcgaagaccagcccgcttagctggtttaccgctgtgggctggtccagtgaagacggagaggcagcggcaggtgaacctcgtgcatgcgtacgtgaaaatcgtgcgcttgttacagctctggttcaggcgcttttggacgaccctcgccgttcgcgcactctgcctcaccgtatgacaagtgcggcacgcgttgcggacggacgcgcacgctatgcgcgtgcagaggcacttttcgcaggagggcgctttttagtcactcgtgaagaagaagtagagctggcggctcgtcccgagttggcgcttctggcatccttggcggctacccctgcaccgccggatcgcttggctgcggggcttgcagaggcgcttggacgcccaggcggtgaccccggggcgcagcgcttcgtagaccaacttgtcacagcacgccttctggtaccaacagaaccggtcgacccccaggatcgtcatccgttgcgcagcttagcaggttggcttcgtcaatggccccaggacgcggaacttgcgcatcgcatcgaacagctggaccgccagaatgcggaactggctgtcactacaggagaacatcgccccgagctgctggctgttttggctgagcgttggcgcctgttgctggccgatgctgggcgcccggtaccgcaagaggccgcacctttatctgttctttcagaggatgttcatgccccggctcctcctcaaccccgcccaggggccgccgatcgtgcagcccttgctgagttgaccgcgttagcagaattgtttgaccacgctcatttaatgcgtcgcgcagcgcgcggacgctttgtcgcgcgttatggcgttggtggagtttgcgatgccccatgggatttcgccgccgacttagccgattcttgggctgacccgactccgcctgacgaattggccgcactgcgtgaagaatttgctaagttgccagaacaagatggagagcttgtattgcccgctgagcgcattcgtgccttggcggcacgtcttcctcactggaccgcagctcgtcccttatcttactcatggtttgttcaacgcggtagtgcggacggcttattgtgtgttaatcacgtatatggcggctgggggcgctttacctcgcgcttcctggacggtctggcccctcaagcggcaacggaagtcgcgcgtcagttacgcagcggtctgggcgctggcgctcgtgccgcgcaaattcgtcccgtaggtggatttaatgctaacttacatccccgtttattagcggacgagatcggaccagatcgtcaatggactggcttggcagagtctgatttggacttggtccacgatcctgtcgatgaccagttgcgcctgcgtttgcgtacaaccggagagttattagacgtactgtatcttggctttctggcaccagtaatgcttccccgtcgtctgggaccgctgctgaacgatcaccccgaaggtgtggtagactttcgtcctttattgccacgcaccacgttagccgcccctggcggtacagtattgcgtaccccccgtttacgccatcgtcacgttgtgcttgcacgccgccgctggttacttcctgccggtgtgctggatgcacttcgtgcagatttagcggccgacgccggacccgatggtgtgccggcagctgctgtagcacgttggcgcgcgcgtcttgatttacctgaacagttgttcttgcatcctgcgccagcggcggcagacccagcaggtacccccggagatgcattcgtggcgcaccttcgtgcacctaaacctcagcccgttgatttgggcaacccacttcacttacgccaccttgctaaatggttgacacgtcatcctcgcggggcagtattggaagaggctcttcccgcgatcgctgggcatcctgagcctactcgcgctgtcgaactggttgttgagacttaccgtccagggcgcggaagtgagcaggcagcaggcgcttttgagtctgttcgtacagcgatcgcggaggaggctcccgacgagtgatgaataactgaataggggatcccgactggcgagagccaggtaacgaatggatccccgagctcgagcaaagcccgccgaaaggcgggcttttctgtccttgagagtcgggcattgtcttcgctccttccggtgggcgcggggcatgactatcgtcgccgcacttatgactgtgttctttatcat

SEQ ID NO: 17 - DNA, 2434 bp, unidentified
  misc_feature 1..2434: note = Synthetic sequence
  source 1..2434: mol_type = other DNA
aaaaccgaattttgctgggtgggctaacgatatccgcctgatgcgtgaacgtgacggacgtaacgaagacaaccacgcattgctgttctgagctaacaccgtgcgtgttgacaattttacctctggcggtgataatggttgcagcaagcaataattttgtttaactttaagaaggagatatacaatgtcagagctgcctgttcttactccagtcgaggctttagcggccacctcggggacggcggtagttcacttgactgagtggacgttgggattggccgcacgtctgtcgcgccatgcgttggcccacccggtacgcttagtcccggttcgcgaggacggggctttaacggtcgtaggtcctgttttagctcccggggcaccggcatgtctggcgtgtgtcgagtaccagcgcctggcgacggcgggcggccgcgttccgtggcaatcgcccgccctggctttgggcgggactggcacacccgcgtttgcggaagccgttacagcgcttgccgcggagcttgcgaaaggtcctgaggccgctgaatcagccgagggggctgggagcccagaagccgcggaatcggcggaaggggctggaagccctgaagctgcggagtctgccgacggtgctggatccccagaggctgcagagtcggccgacggtgctggttcccccgaagccgcagagagcgccgatggcgccggatctccagaagcggctgaaagcgcagacggagcaacggttcatgttgtccacggaggccgtgccacatggtcgacacatcgtgtacgccctgttggaggttgcgaagtgtgccgccctctgcctccggacacgccggaggcggcgcgcttgcccgcaacaccccgcccccttccggatccagcagtgctgcgcggccccaacgatcgcacggatgcaggccagctgcgcgccgagctgtatgatgagcgcttcggaccagtacgccgcctgtttcgcactgaggattcggcgtttgcgctgacgacagcttgggttacggatgggcgtgctttagatgacggcggttatggacgcgctgcagatttccgctcgagcgagcgtgttgcactttttgaagccgtagagcgccacgccggtatgcgcccccttggccgtcgcaccgtcttacgcgcaagttatgcagaattggctcgtgagttaggaccggatgcagtccttgacccggcccgcctgggtctgccagatgatccccaccagggccacccgacaccggccactgccccctacacacctgaattagtattggattgggtgcacgggtggagcctgacgcgccgccgtcctgtggctgtaccggaacgtgtcgcctattgggaagtaccagggcgcgatcgtccgcgtgtggtgtatgagtcctcaaatgggtgtggcttggggaacagcccccaagaagcggcgctttacggtttatttgaggtcgcagaacgcgacgctttcctgatggcctggtatgcgcgcactccactgccgggtgtcgcggttcccactgaagatcctcaaattgccgagcttgctgatcgcgctgaattattcggctaccacttgactttgttggacgcgacgaacgatttaggggtccctgccgtcattgcgttgtgccgccatcgcggagatcatcccgatgctcctcgtaccctgttggccgcaggcgcgcaccatgatccgcgtacagccatccgctctgcagtggctgaagtggtaacaaatgtccaagaggccccgcatcgttctactgcgcccggcggcccccgcgatccacagcgccttcgcccgatgcttgagcgccctgagttagtcgtgtcattggacgatcatgtgggcctgaacacgctgcctgaagctcagccacgtttggacttcctttttgcgggaccaccaccggtcccgtggacagaacgttggccaggtgatcccgaaccggttacggatttaaccgaccttcttgaacgcactgtaacacgcttagctggagaagatttggaggttctggttgtcactcaagatgaacccggtgtgcgcgatcgcttaggtttgcactgtgcaaaagttgtcgtgcccggtactttgccaatgacgtttggacatgctaaccgtcgtacgcgcggattatcacgcttattggaagtgccctatcgtttaggtcgtactccggcccctttgcgccacgacgagctgcccttgcacccgcatccgttcccctgatgaataactgaataggggatcccgactggcgagagccaggtaacgaatggatccccgagctcgagcaaagcccgccgaaaggcgggcttttctgtccttgagagtcgggcattgtcttcgctccttccggtgggcgcggggcatgactatcgtcgccgcacttatgactgtgttctttatcat

SEQ ID NO: 18 - DNA, 1903 bp, unidentified
  misc_feature 1..1903: note = Synthetic sequence
  source 1..1903: mol_type = other DNA
caaccacgcattgctgttctgagctaacaccgtgcgtgttgacaattttacctctggcggtgataatggttgcagcaagcaataattttgtttaactttaagaaggagatatacaatgacaacccatgccctgccggctacgacatggcactcactgcatttggcacttcccctgcctgcccgtgaagccgacgcatttcttacggaggatttggctccgctgatggacggtctggcaggcaccgattggtttttcatccgctacggagagggggggccccatctgcgcatccgtcatcgtggcccgggccctgcgccagctagccttgcagcagacttgacccgcttggctacacgtcgtaccgcgcccgatggcccattcgcggatggtcatggaactgtcacagaggtaccatatgaacccgaaacagagcgctatggtggagctgccttacttccgattgctgaggaagtgtttacgcactctacccgtgccgctgtgcgtgcgttacacgccttaggagcggctcctgagaagcgtctgcagctggcactggatttagcgcacaccactgcttatgcgttgggcctggatgagctggcggcatcacgttggcttcgccgtcatgcggcagcatggcgctgggttactgaatttcgcccactgcctggcgcagctgtacatacccgtgtcaacacggtattcgcacgccaacgtgaaactcttgcacgtcgcgctcgtgaacttcgcgccgcacttgatgcaggcaccgcgtctccctggctgggagattgggccgcccgcgccgccgaagctgcagcgcgtatgcgcgctgtagctgcggcagaagctgccgctcctgcgagcgctgcagaggaggctgaagagcgcctggaatggatctgggcaagtcagttgcatatgttgttcaatcgcttaggcgtaggtccagacgaagaacgcgccgtgtgtcgtttagcggctcgcacattgctggagacaggccaaccgttcacattcttcccagccgatcatcatgcccccgaccaccagtatctggagcgttcgaaatttcaaatcggtcgcggagaggatactgctttgcgtgatttaccccatgatcctgcaccctcccctcgtccgggcgacttacccttacccgcagatcccttgcccccggtgacgttggcggatgcgttgcgcactcgccgttcaacccgcggccctttaacgggcccacttacggccggtgcgctggggggactgttgtggagtgccttcgctccagctcctgacacagggcatcgcccgtatccgagtgcgggggcccttcatactgttcgccttcgcctgcttgccctggcggttgatgggctgcccgcaggtacgtaccactgtttacccgagcaccgcagcctgcgccccatcgggcccgcaccggcgttggatgacttgaaggcgttgtctagctatctgagccgcccggcagaggaccccgacgcgattggtgtagatcgcgcacccgcggtcttagccgtttatcttgacttagcacgtctgcgtcgtcgctacggacttcgcgcgttacgccttggcgtacttgaggcaggccatttggcacagaacttgctgttgacctctgctgcgtttggtctggggacaacaccattagggggcctgcaggatgaccttgcacatgaattgttaggtcttgatgatttaggcgagcccatccaatacctgttaccgctggggcgtccggggaccgtgccggtgattatggagtgatgaataactgaataggggatcccgactggcgagagccaggtaacgaatggatccccgagctcgagcaaagcccgccgaaaggcgggcttttctgtccttgagagtcgggcattg

SEQ ID NO: 19 - DNA, 1583 bp, unidentified
  misc_feature 1..1583: note = Synthetic sequence
  source 1..1583: mol_type = other DNA
caaccacgcattgctgttcataaatgtgagcggataacattgacattgtgagcggataacaagatactgagcacagcaagcaataattttgtttaactttaagaaggagatatacaatgaaaatcgaagaaggtaaactggtaatctggattaacggcgataaaggctataacggcctcgctgaagtcggtaagaaattcgagaaagataccggaattaaagtcaccgttgagcatccggataaactggaagagaaattcccacaggttgcggcaactggcgatggccctgacattatcttctgggcacacgaccgctttggtggctacgctcaatctggcctgttggctgaaatcaccccggacaaagcgttccaggacaagctgtatccgtttacctgggatgccgtacgttacaacggcaagctgattgcttacccgatcgctgttgaagcgttatcgctgatttataacaaagatctgctgccgaacccgccaaaaacctgggaagagatcccggcgctggataaagaactgaaagcgaaaggtaagagcgcgctgatgttcaacctgcaagaaccgtacttcacctggccgctgattgctgctgacgggggttatgcgttcaagtatgaaaacggcaagtacgacattaaagacgtgggcgtggataacgctggcgcgaaagcgggtctgaccttcctggttgacctgattaaaaacaaacacatgaatgcagacaccgattactccatcgcagaagctgcctttaataaaggcgaaacagcgatgaccatcaacggcccgtgggcatggtccaacatcgacaccagcaaagtgaattatggtgtaacggtactgccgaccttcaagggtcaaccatccaaaccgttcgttggcgtgctgagcgcaggtattaacgccgccagtccgaacaaagagctggcaaaagagttcctcgaaaactatctgctgactgatgaaggtctggaagcggttaataaagacaaaccgctgggtgccgtagcgctgaagtcttacgaggaagagttggcgaaagatccacgtattgccgccaccatggaaaacgcccagaaaggtgaaatcatgccgaacatcccgcagatgtccgctttctggtatgccgtgcgtactgcggtgatcaacgccgccagcggtcgtcagactgtcgatgaagccctgaaagacgcgcagactaattcgagctcgaacaacaacaacaataacaataacaacaaccccatgagcgagaatctttattttcagggccaaatgattaagcattttcattttaataaactgtcttctggtaaaaaaaataatgttccatctcctgcaaagggggttatacaaataaaaaaatcagcatcgcaactcacaaaaggtggtgcaggacatgtgcctgagtattttgtggggattggtacacctatatctttctatggctgatgaataactgaataggggatcccgactggcgagagccaggtaacgaatggatccccgagctcgagcaaagcccgccgaaaggcgggcttttctgtccttgagagtcgggcattg

SEQ ID NO: 20 - DNA, 1484 bp, unidentified
  misc_feature 1..1484: note = Synthetic sequence
  source 1..1484: mol_type = other DNA
aaaaccgaattttgctgggtgggctaacgatatccgcctgatgcgtgaacgtgacggacgtaacgaagacaaccacgcattgctgttcataaatgtgagcggataacattgacattgtgagcggataacaagatactgagcacagcaagcaataattttgtttaactttaagaaggagatatacaatgaaaatcgaagaaggtaaactggtaatctggattaacggcgataaaggctataacggcctcgctgaagtcggtaagaaattcgagaaagataccggaattaaagtcaccgttgagcatccggataaactggaagagaaattcccacaggttgcggcaactggcgatggccctgacattatcttctgggcacacgaccgctttggtggctacgctcaatctggcctgttggctgaaatcaccccggacaaagcgttccaggacaagctgtatccgtttacctgggatgccgtacgttacaacggcaagctgattgcttacccgatcgctgttgaagcgttatcgctgatttataacaaagatctgctgccgaacccgccaaaaacctgggaagagatcccggcgctggataaagaactgaaagcgaaaggtaagagcgcgctgatgttcaacctgcaagaaccgtacttcacctggccgctgattgctgctgacgggggttatgcgttcaagtatgaaaacggcaagtacgacattaaagacgtgggcgtggataacgctggcgcgaaagcgggtctgaccttcctggttgacctgattaaaaacaaacacatgaatgcagacaccgattactccatcgcagaagctgcctttaataaaggcgaaacagcgatgaccatcaacggcccgtgggcatggtccaacatcgacaccagcaaagtgaattatggtgtaacggtactgccgaccttcaagggtcaaccatccaaaccgttcgttggcgtgctgagcgcaggtattaacgccgccagtccgaacaaagagctggcaaaagagttcctcgaaaactatctgctgactgatgaaggtctggaagcggttaataaagacaaaccgctgggtgccgtagcgctgaagtcttacgaggaagagttggcgaaagatccacgtattgccgccaccatggaaaacgcccagaaaggtgaaatcatgccgaacatcccgcagatgtccgctttctggtatgccgtgcgtactgcggtgatcaacgccgccagcggtcgtcagactgtcgatgaagccctgaaagacgcgcagactcgtatcaccaagtaatgaataactgaataggggatcccgactggcgagagccaggtaacgaatggatccccgagctcgagcaaagcccgccgaaaggcgggcttttctgtccttgagagtcgggcattgtcttcgctccttccggtgggcgcggggcatgactatcgtcgccgcacttatgactgtgttctttatcat

SEQ ID NO: 21 - protein, 39 aa, unidentified
  REGION 1..39: note = Synthetic sequence
MTKRTYETPVLVSAGSFARRTGSGSPKAARDPFGRRWLP

SEQ ID NO: 22 - protein, 6 aa, unidentified
  REGION 1..6: note = Synthetic sequence
GGGGSS

SEQ ID NO: 23 - protein, 4 aa, unidentified
  REGION 1..4: note = Synthetic sequence
GGSG

SEQ ID NO: 24 - protein, 10 aa, unidentified
  REGION 1..10: note = Synthetic sequence
GGSGGGGSGG

SEQ ID NO: 25 - protein, 20 aa, unidentified
  REGION 1..20: note = Synthetic sequence
SFAVHGIWETYLRDQMNKCP

SEQ ID NO: 26 - protein, 8 aa, unidentified
  REGION 1..8: note = Synthetic sequence
DYKDDDDK